(19) United States
(12) Patent Application Publication
(10) Pub. No.: US 2005/0165004 A1
(43) Pub. Date: Jul. 28, 2005

(54) Bronchorelaxing compounds

(76) Inventors: Staffan Skogvall, Lund (SE); Henrik Bjork, Lund (SE); Per Magnus Berglund, Lund (SE); Maria Fanny Dalence Guzman, Lund (SE); Olov Sterner, Malmo (SE)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/761,323

(22) Filed: Jan. 22, 2004

(51) Int. Cl. A61K 31/55; A61K 31/47; C07D 217/06; C07D 223/16

(52) U.S. Cl. 514/217.01; 514/310; 540/594; 546/146

(57) ABSTRACT

A compound of the general formula (I) including its pharmaceutically acceptable acid addition salts 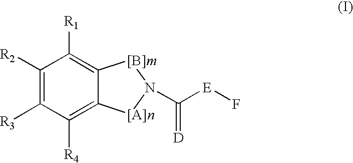
wherein A is CHR₉, wherein R₉ is H, C₁-C₆ alkyl; n is 1-3; B is CHR₁₀, wherein R₁₀ is H, C₁-C₆ alkyl; m is 1 or 2; D is O or S or optionally NR₁₆, wherein R₁₆ is H, C₁-C₆ alkyl or C₂-C₆ acyl; E is CR₁₁R₁₂ or NR₁₃, wherein R₁₁ and R₁₂ are, independent of each other, H or C₁-C₆ alkyl, R₁₃ is H or C₁-C₆ alkyl; F is C₁-C₁₈ alkyl which may be mono- or di-unsaturated and/or substituted, is useful in treating and preventing pulmonary disease characterized by bronchoconstriction. Also disclosed are pharmaceutical compositions comprising the compound and methods for their manufacture.

### FIELD OF THE INVENTION

The present invention relates to novel bronchorelaxing compounds, pharmaceutical compositions comprising such compounds, and a method of treating or allevating conditions accompanied by bronchoconstriction.

### BACKGROUND OF THE INVENTION

Airway obstruction, accompanied by an increase in the contractile state of the bronchial smooth muscle, is prominent in a number of diseases of the respiratory apparatus, in particular asthma, chronic obstructive pulmonary disease (which comprises chronic bronchitis and emphysema), bronchiectasis, cystic fibrosis, bronchiolitis and bronchopulmonary dysplasia. Bronchoconstriction may be caused by a number of factors that affect the bronchi and other parts of the respiratory apparatus independent of each other or in combination. The available means for treating or preventing bronchoconstriction are insufficient in many respects. Thus new compounds that exert a relaxing effect on constricted bronchi are much in need.

### OBJECTS OF THE INVENTION

It is an object of the present invention to provide a compound for treating or preventing bronchoconstriction and for use in treating diseases such as asthma, in which bronchoconstriction is prominent.

It is another object of the present invention to provide a pharmaceutical composition comprising said compound.

Still another object of the present invention is to provide a method for treating or preventing bronchoconstriction by administration of such compound to a person in need.

Further objects of the invention will become apparent from the following summary of the invention, the description of preferred embodiments thereof, and the appended claims.

### SUMMARY OF THE INVENTION

According to the present invention is disclosed a compound of the general formula (I) including its pharmaceutically acceptable acid addition salts 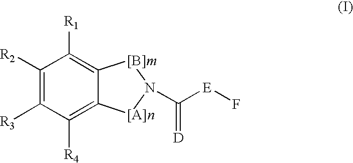
wherein
R₁-R₄ are, independent of each other H; C₁-C₆ alkyl; halogen; NR₅R₆, wherein R₅ and R₆ are, independent of each other, H, C₁-C₆ alkyl, C₂-C₆ acyl; OR₇, wherein R₇ is H, C₁-C₆ alkyl or C₂-C₆ acyl; CN; COR₈, wherein R₈ is H, C₁-C₆ alkyl or C₁-C₆ alkoxy;
A is CHR₉, wherein R₉ is H, C₁-C₆ alkyl;
n is 1-3;
B is CHR₁₀, wherein R₁₀ is H, C₁-C₆ alkyl;
m is 1 or2;
D is O or S or optionally NR₁₆, wherein R₁₆ is H, C₁-C₆ alkyl or C₂-C₆ acyl;
E is CR₁₁R₁₂ or NR₁₃, wherein R₁₁ and R₁₂ are, independent of each other, H or C₁-C₆ alkyl and wherein R₁₃ is H or C₁-C₆ alkyl;
F is C₁-C₁₈ alkyl, which may be mono- or di-unsaturated and/or substituted by alkyl, aryl, substituted aryl, heteroaryl, substituted heteroaryl, wherein, independent of each other, said C₁-C₁₈ and said alkyl, aryl, substituted aryl, heteroaryl, substituted heteroaryl substituent(s) is optionally further substituted by one to three substituents independently selected from F, Cl, Br;
   with the proviso that,
if R₁ and R₂ are H, n is 2, m is 1, D is S, E is NH, F is 2-(4-chlorophenyl)ethyl or octyl, R₃ and R₄ are not both OH or OH and OCH₃;
if R₁ and R₄ are H, n is 2 or 3, m is 1, D is S, E is NH, F is 2-(4-chlorophenyl)ethyl or octyl, R₂ and R₃ are not both OH or OH and OCH₃.

In the compound of the general formula (I) R₉ and R₁₀ are preferably H. Preferably R₁₁ is also H, independent of whether R₉ and R₁₀ are H. Preferably R₁₂ is also H, independent of whether one or more of R₉, R₁₀, R₁₁ are H. Preferably R₁₃ is also H, independent of whether one or more of R₉ , R₁₀, R₁₁, R₁₂ are H.

In the compound of the general formula (I) it is particularly preferred for R₁₁ and R₁₃ to be H, in particular if R₉ and R₁₀ are H; in such case it is also preferred for R₁₂ to be H.

The pharmaceutically acceptable addition salts as mentioned hereabove comprise the therapeutically active non-toxic addition salt forms which the compounds of the general formula (I) are able to form. They can conveniently be obtained by treating the base form with appropriate inorganic, such as, for instance, hydrochloric acid, hydrobromic acid, sulfuric acid, nitric acid, phosphoric acid and the like, or with appropriate organic acids, such as, for instance, acetic, propanoic, methanesulfonic, benzenesulfonic, lactic, malic, citric, tartaric, succinic, maleic acid and the like. The term acid addition salt also comprises the hydrates and solvent addition forms, such as hydrates and alcoholates, which the compounds of the general formula (I) are able to form.

According to a first preferred aspect of the invention, in the compound of the general formula (I), F is ω-(C₁-C₃)R₁₄, wherein R₁₄ is substituted or non-substituted aryl or heteroaryl. Preferably R₁₄ is mono-, di- or trisubstituted aryl or mono-, di- or trisubstituted heteroaryl, wherein said mono-, di- or trisubstitution is by any of C₁-C₆ alkyl; aryl; heteroaryl; halogen; hydroxy, C₁-C₃ alkoxy; methylenedioxy; nitro; cyano; carboxy C₁-C₆ alkyl; R₁₅CO, wherein R₁₅ is H, C₁-C₆ alkyl, aryl; amino; alkylamino, dialkylamino; fully or partially fluorinated C₁-C₆ alkyl; with the proviso that, in case of di- or trisubstitution, the substituents are same or different. Even more preferred is the selection of at least one substituent from C₁-C₆ alkyl, aryl, F, Cl, Br, methyl, trifluoromethyl, nitro, methoxy. Also preferred is the selection of at least two substituents from C₁-C₆ alkyl, aryl, F, Cl, Br, methyl, trifluoromethyl, nitro, methoxy.

According to a second preferred aspect of the invention, in the compound of the general formula (I) at least one of R₁-R₄ is halogen; preferably said last of R₁-R₄ is R₁ or R₄. The preferred halogen is chloro.

According to a third preferred aspect of the invention, in the compound of the general formula (I) at least one of R₁-R₄ is halogen, preferably said at least one of R₁-R₄ being R₁ or R₄, whereas the preferred halogen is chloro or bromo, preferably chloro, and whereas, in addition to said at least one halogen, at least one of remaining R₁-R₄ is hydroxy or methoxy.

According to a fourth preferred aspect of the invention, in the compound of the general formula (I) at least two of R₁-R₄ are halogen, in particular chloro or bromo, more preferred chloro, preferably R₁ and/or R₄; in addition to said at least two halogens at least one, preferably two of remaining R₁-R₄ are, independent of each other, hydroxy or methoxy or methylenedioxy.

According to a fifth preferred aspect of the invention, in the compound of the general formula (I), at least one, preferably at least two of R₁ to R₄ are, independent of each other, hydroxy or methoxy or methylenedioxy, more preferred hydroxy, even more preferred hydroxy pertaining to a pyrocatechol structure which may be dimethylated. Also preferred is one of R₁ to R₄ to be hydroxy and another methoxy, preferably in an ortho relationship.

According to a sixth preferred aspect of the invention, in the compound of the general formula (I), at least one of R₁ to R₄ is hydroxy or methoxy and at least another of R₁ to R₄ is chloro or bromo, preferably chloro, and wherein said hydroxy or methoxy and said chloro or bromo are in an ortho relationship.

According to a seventh preferred aspect of the invention, in the compound of the general formula (I), at least two of R₁-R₄ are methoxy or comprised by methylenedioxy.

According to an eight preferred aspect of the invention, in the compound of the general formula (I), it is preferred for D to be S or O, most preferred to be S.

According to a ninth preferred aspect of the invention, the following compounds comprised by the general formula (I) are preferred:

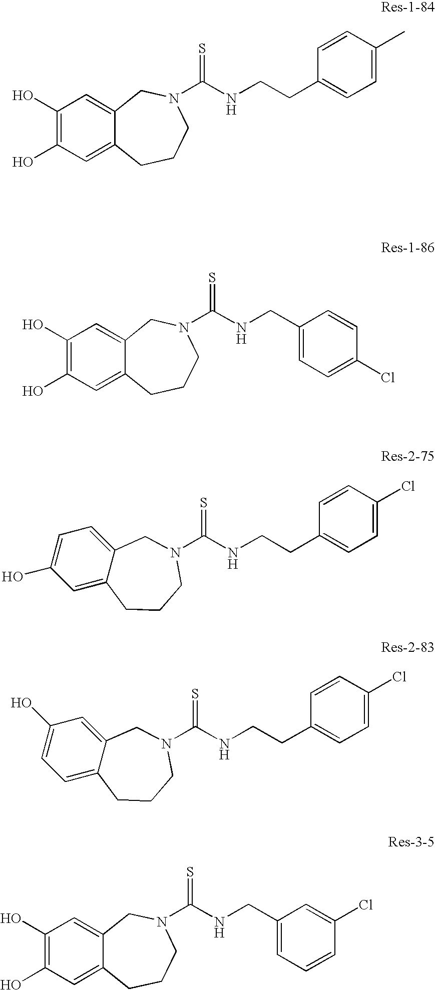

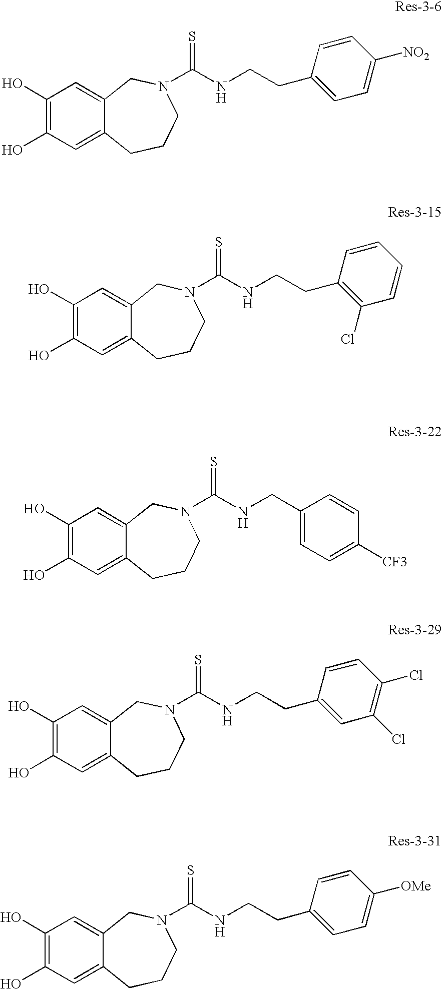

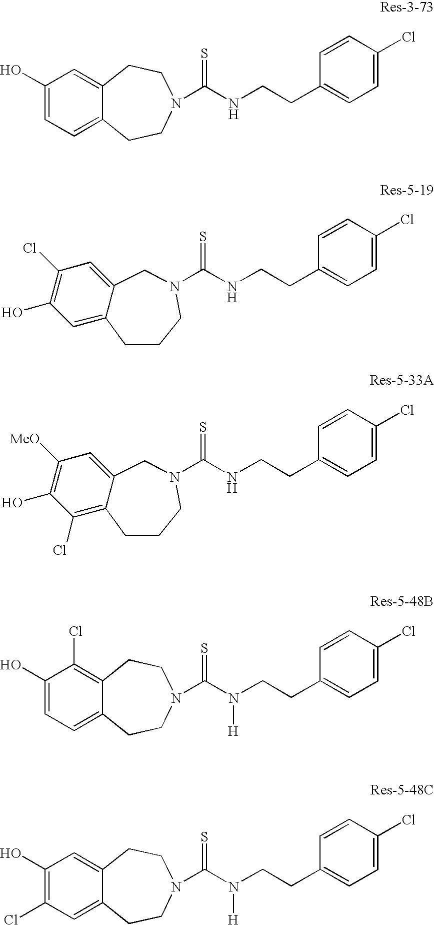

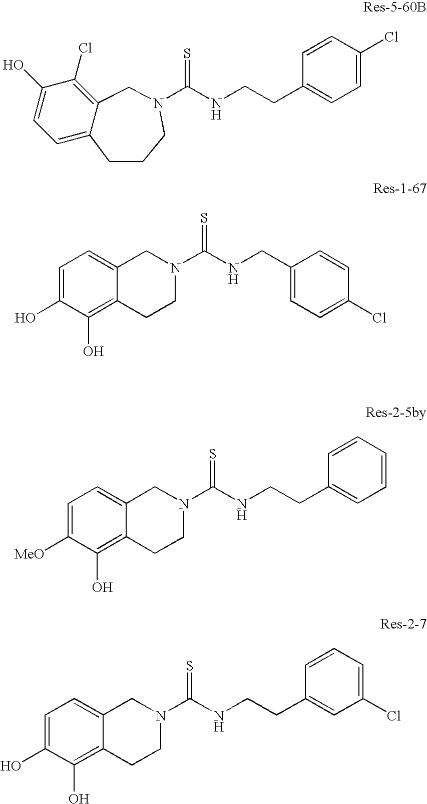

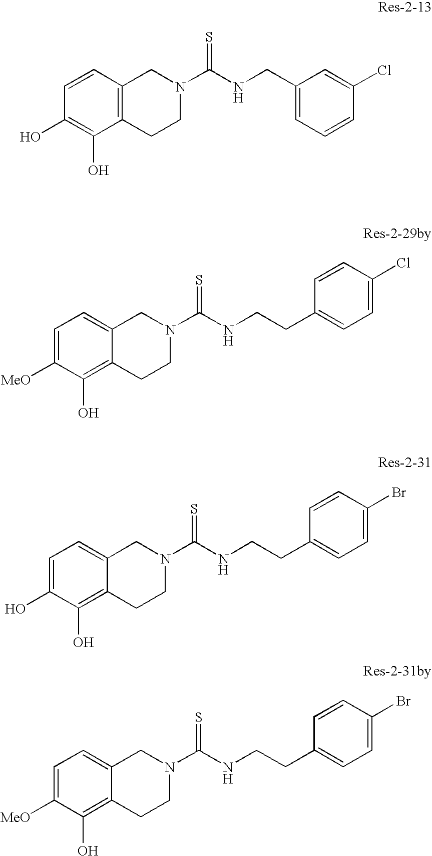

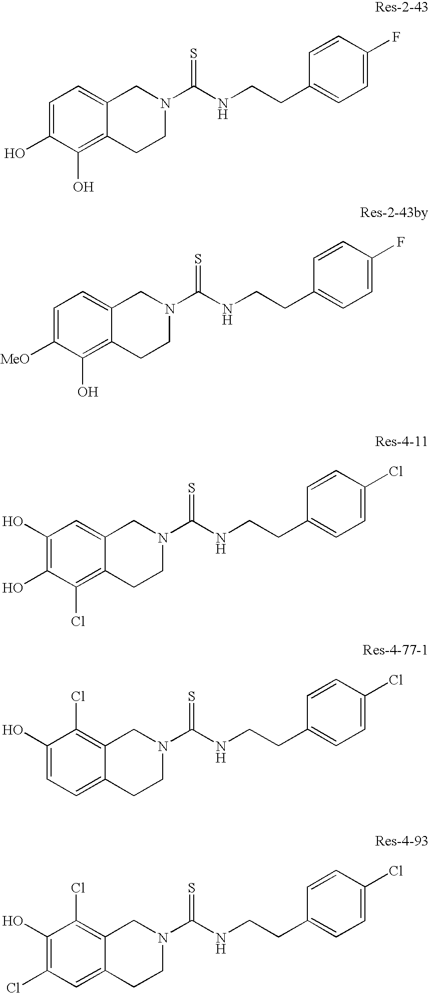

According to a tenth preferred aspect of the invention, the following compounds comprised by the general formula (I) are even more preferred:

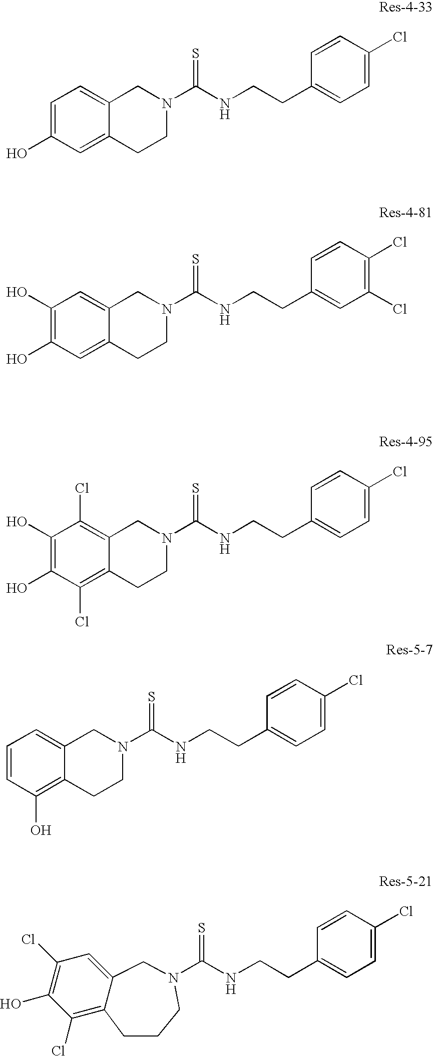

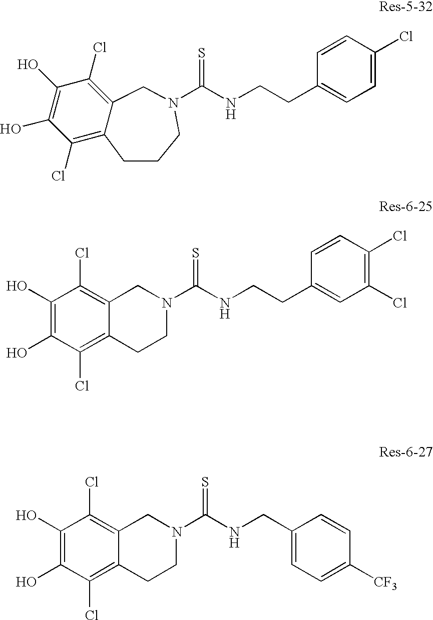

According to an eleventh aspect of the invention the most preferred compound is

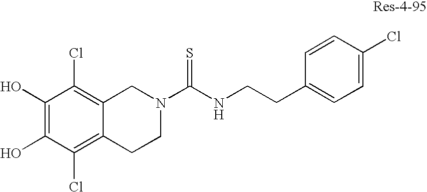

The term “C₁-C₆ alkyl” comprises straight and branched chain alkyl, such as methyl, ethyl, propyl, isoproyl, butyl, isobutyl, t-butyl, pentyl, 2-methylbutyl, hexyl, 2-methylpentyl.

The term “C-C₆ acyl” comprises straight and branched chain acyl, such as acetyl, propionyl, butyryl, iso-butyryl.

The term “halogen” comprises F, Cl, Br, I.

The compounds of the invention have been tested for their bronchoconstriction-inhibiting or bronchorelaxing effect in a model comprising a human bronchus preparation. The model is described in detail in the Preferred Embodiments section. Particularly preferred compounds according to the invention are those which exhibit in this model a bronchorelaxing effect which is about the same or even better than that of capsazepine on a weight/weight basis. Most preferred compounds according to the invention are those which exhibit in this model a bronchorelaxing effect which is superior to that of capsazepine on a weight/weight basis

The compounds of the present invention and their pharmaceutically acceptable acid addition salts can be used in the treatment of diseases in which the constriction of the bronchi is of importance, such as asthma. The present compounds may block bronchoconstriction agonist-induced contractions of bronchial tissues.

The compounds of the invention can therefore be used as medicines against above-mentioned diseases or in their prevention. Said use as a medicine or method of treatment comprises the systemic administration to patients of an amount effective to combat bronchoconstriction.

The compounds of the invention can be formulated into various pharmaceutical forms for administration purposes. Said pharmaceutical forms or compositions are deemed novel and consequently constitute another aspect of the present invention. Also the preparation of said compositions constitutes a further aspect of the present invention. To prepare the pharmaceutical compositions of this invention, an effective amount of the particular compound, including in acid addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, preferably, for administration orally, rectally, percutaneously, or by parenteral injection. Particularly preferred is administration by inhalation.

For example, in preparing the compositions in oral dosage form, any of the usual pharmaceutical media may be employed such as, for example, water, glycols, oils, alcohols and the like in the case of oral liquid preparations such as suspensions, syrups, elixirs and solutions: or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier option-ally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on or as an ointment. Acid addition salts of the compound of general formula (I) due to their increased water solubility over the corresponding base form, are obviously more suitable in the preparation of aqueous compositions. It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof. Administration by inhalation will allow a high proportion of the delivered dose to reach the site of action, that is, the bronchi and the lung in general. Inhalation may be by the oral or the nasal route. Conventional pulmonary applicators may be employed, such as pressurized spray containers containers suitable propellants for aerosols and powder spray devices for preparations in form of fine powders. Pharmaceutical compositions suitable for administration by the inhalation route are known in the art. The compound is dissolved in a suitable vehicle or employed as a fine powder, such as a micronized powder of a particle size from about 2 μm to about 20 μm. An indicated daily dose for administration by inhalation will be 10 times and more lower than the oral dose. Satisfactory doses, preferably metered by using a device capable of metering, or by single doses of predetermined size, can easily be determined by experimentation.

In view of the usefulness of the compounds of the invention in the treatment of diseases in which bronchoconstriction is prominent, it is evident that the present invention provides a method of treating warm-blooded animals suffering from such diseases, said method comprising the systemic administration of a pharmaceutically effective amount of a compound of formula (I) or a pharmaceutically acceptable acid addition salt thereof in admixture with a pharmaceutical carrier. Those of skill in the treatment of diseases in which bronchoconstriction is an important factor could easily determine the effective amount. In general it is contemplated that an effective amount would be from 0.01 mg/kg to 4 mg/kg body weight, preferably from 0.04 mg/kg to 2 mg/kg body weight.

The exact dosage and frequency of administration depends on the particular compound of formula (I) used, the particular condition being treated, the severity of the condition being treated, the age, weight and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention. The effective daily amount ranges mentioned hereinabove are therefore guidelines only and are not intended to limit the scope or use of the invention.

Unless otherwise stated all parts in this specification are by weight.

### SHORT DESCRIPTION OF THE FIGURES

The invention will now be explained in greater detail by reference to a number of preferred but not limiting embodiments illustrated in a drawing in which

FIGS. 1-6 are charts in which the bronchorelaxing effect of compounds of the invention is compared with that of capsazepine, the bronchorelaxing effect of some other prior art compounds also being shown;

FIG. 7 is a time v. force diagram of the determination of the bronchorelaxing effect of capsazepine as an exemplary test compound. At (B) the preparation is mechanically tensioned by a selected force.

### DESCRIPTION OF PREFERRED EMBODIMENTS OF THE INVENTION

### A. SYNTHESIS OF SUBSTITUTED THIOUREA COMPOUNDS OF THE INVENTION (D=S)

### EXAMPLE 1

### Synthesis of 1,3,4,5-tetrahydro-2H-2-benzazepine-2-carbothioamides and 1,2,4,5-tetrahydro-3H-3-benzazepine-3-carbothioamides

1,3,4,5-Tetrahydro-2H-2-benzazepine-2-carbothioamides and 1,2,4,5-tetrahydro-3H-3-benzazepine-3-carbothioamides of the invention were synthesized starting from commercially available 1- or 2-tetralones. The tetralones were converted to the corresponding benzazepinones via a Schmidt reaction. Benzazepinones were then reduced to the corresponding benzazepines with borane. In some cases, the aromatic ring of benzazepines was chlorinated using sulfuryl chloride. The methoxyarylethers were cleaved under reflux in concentrated hydrobromic acid. The protonated benzazepines were coupled to isothiocyanates, which were synthesized from the corresponding amines by reaction with thiophosgene, to give 1,3,4,5-tetrahydro-2H-2-benzazepine-2-carbothioamides or 1,2,4,5-tetrahydro-3H-3-benzazepine-3-carbothioamides. The reaction paths are illustrated in Reaction Schemes A and B.

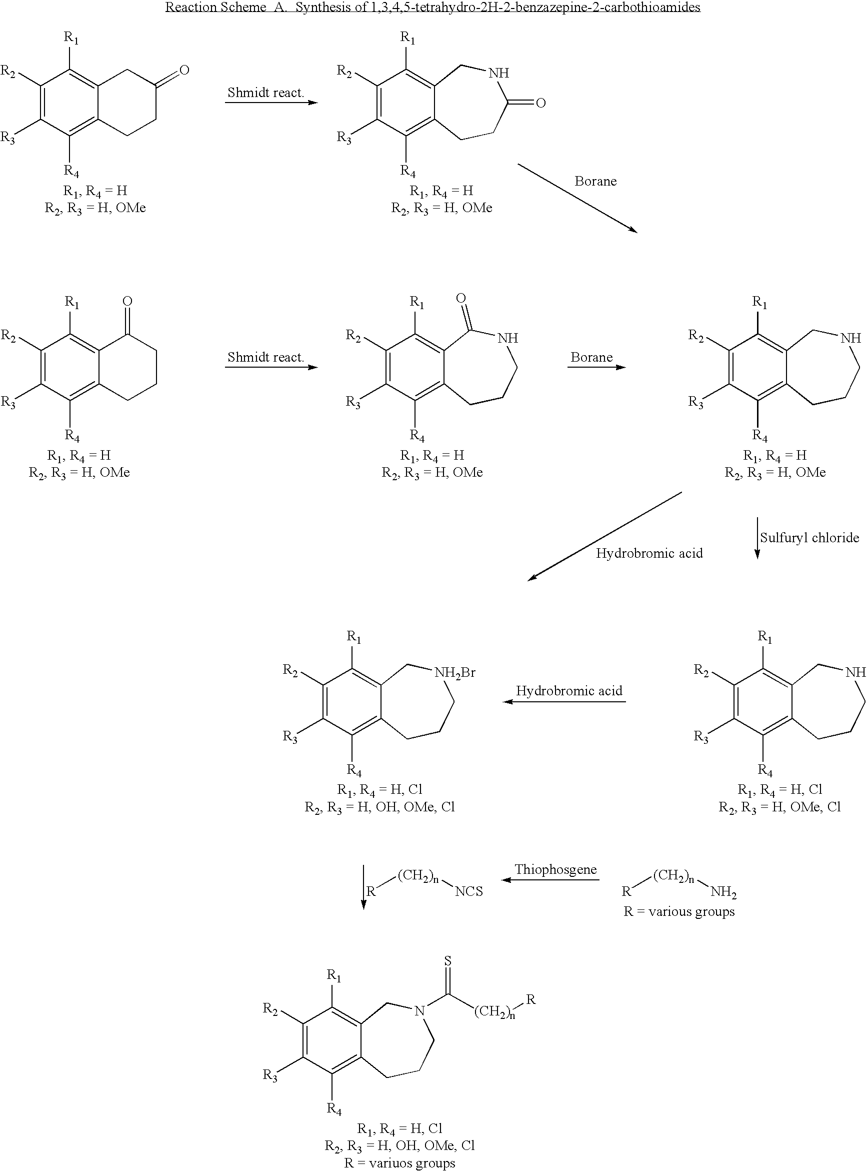

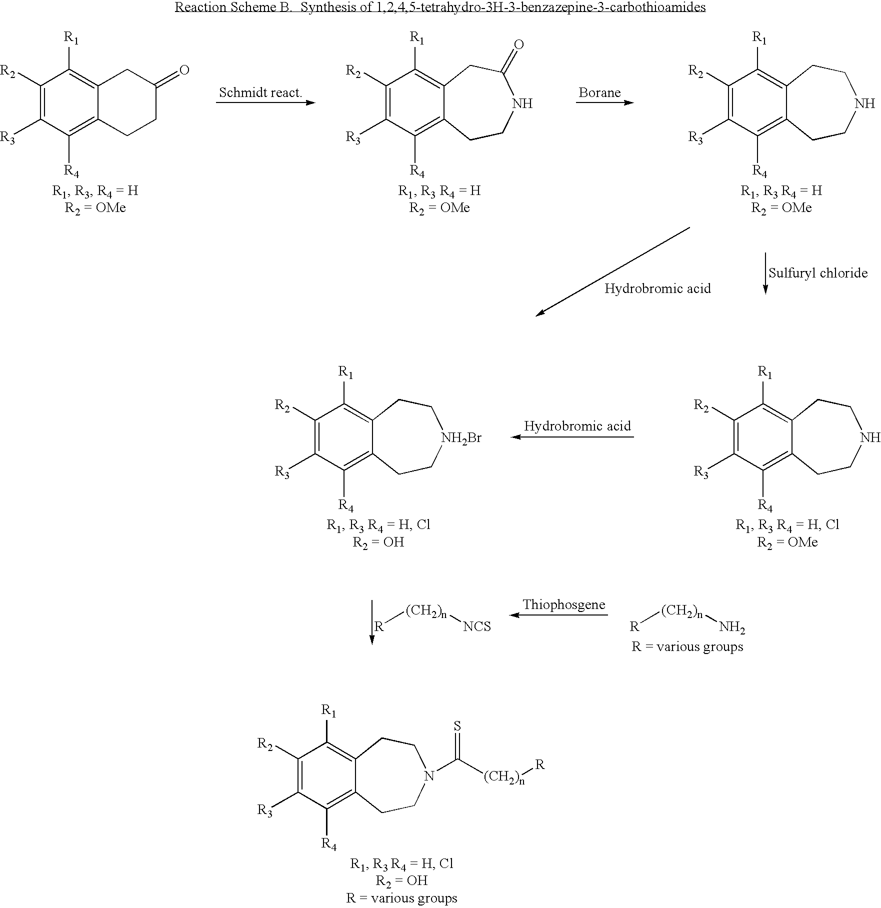

### EXAMPLE 2

### Synthesis of 3,4-dihydroisoquinoline-2(1H)-carbothioamides

3,4-Dihydroisoquinoline-2(1H)-carbothioamides of the invention were synthesized starting from 2-(methoxyphenyl)-ethylamines. The amines were cyclisized with modified Pictet-Spengler conditions and Boc-protected to simplify purification. The cyclic amines were chlorinated in some cases using sulfuryl chloride and Boc-protected to simplify purification The methoxyarylethers were cleaved under reflux in concentrated hydrobromic acid, which also cleaved the Boc-group. The protonated amines were coupled to isothiocyanates, which were synthesized from the corresponding amines by reaction with thiophosgene, to give 3,4-dihydroisoquinoline-2(1H)-carbothioamides. The reaction paths are illustrated in Reaction Scheme C.

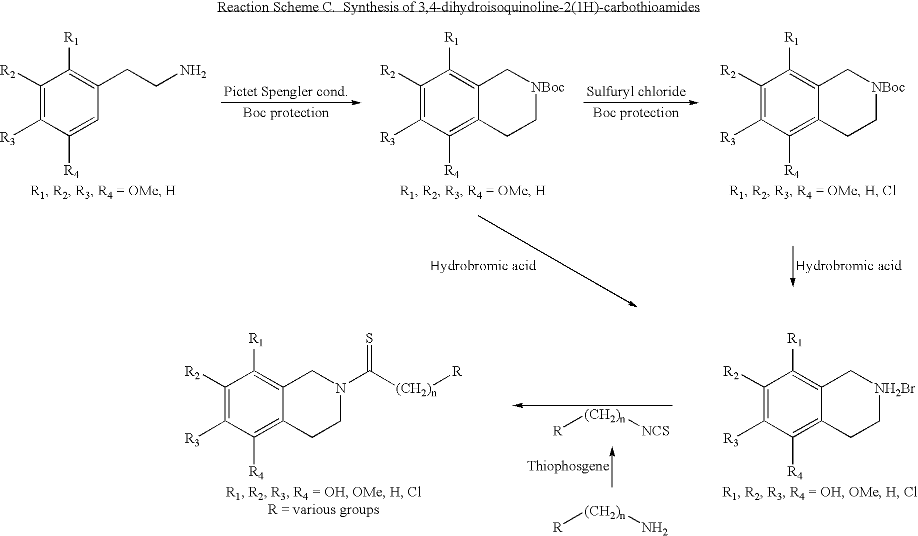

### EXAMPLE 3

### Synthesis of Tetrahydro-Benzazepinones

The tetralone (1 eq.) was dissolved in methanesulfonic acid. The solution was cooled on an ice bath and NaN₃ (1.3 eq.) was added over a period of 30 minutes. The mixture was stirred at room temperature for 18 hours. It was then cooled on an ice bath and a saturated solution of NaHCO₃ was added until slight basicity. The aqueous phase was extracted with CH₂Cl₂. The organic phase was dried (MgSO₄) and concentrated. The residue was chromatographed on silicagel (gradient elution, 40-100% EtOAc in CH₂Cl₂). The tetralone starting materials and the corresponding benzazepinones are listed in Table 1.

TABLE 1
Synthesis of tetrahydro-benzazepinones

| | | | Yield/Isomer |
|---|---|---|---|
| Tetralone | Benzazepinone | | Ratio |
| 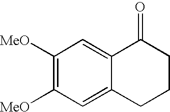 | 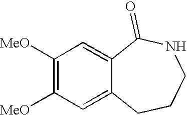 | 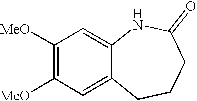 | 65% 4:1 |
| 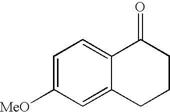 | 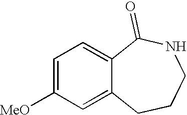 | 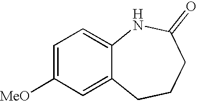 | 60% 6.1 |
| 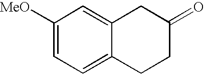 | 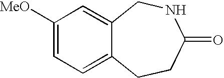 | 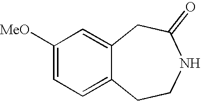 | 63% 1:2 |

### EXAMPLE 4

### Synthesis of Tetrahydro-Benzazepines

The tetrahydro-benzazepinone (1 eq.) was suspended in THF (dry) and the suspension was cooled on an ice bath under nitrogen. A solution of borane in THF (3 eq.) was then added dropwise. The reaction mixture was then refluxed (70° C.) overnight. After, the mixture was cooled on an ice bath and a large excess of MeOH and 5N HCl solution (equal amounts) were added. The solution was heated to 90° C. for two hours. Solvents were then evaporated. Purification was done by re-crystallization of the hydrochloride from a mixture of CH₂Cl₂ and MeOH. The benzazepinone starting materials and the corresponding benzazepines are listed in Table 2.

TABLE 2
Synthesis of benzazepines

| Benzazepinone | Benzazepine Hydrochloride | Yield |
|---|---|---|
| 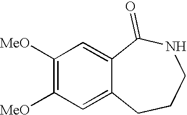 | 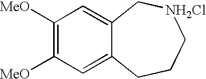 | 85% |
| 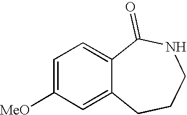 | 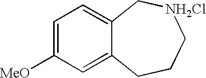 | 94% |
| 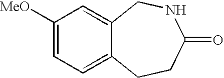 | 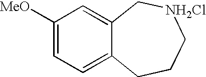 | quantitative |
| 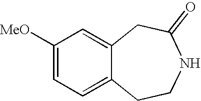 | 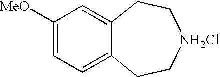 | quantitative |

### EXAMPLE 5

### Synthesis of methoxy-1,2,3,4-trtrahydroisoquinilines

2-(Methoxyphenyl)ethylamine (1 eq.), paraformaldehyde (5 eq.) and MgSO₄ (3 eq.) were suspended in CH₂Cl₂ (dry). After stirring for 2 hours the solid was filtered off. The filtrate was concentrated. The residue was dissolved in trifluoroacetic acid (dry) and refluxed under nitrogen over night. The mixture was poured into a mixture of ice and water. The water phase was made basic with NaOH (6M) and extracted with CH₂Cl₂. The organic phase was dried (MgSO₄) and concentrated. The remaining oil was dissolved in THF. To this solution di-tert-butyldicarbonate (1.2 eq.) and triethylamine (3 eq.) were added. The mixture was stirred for 3 hours and then concentrated. The residue was dissolved in EtOAc and washed with Na₂CO₃ (sat.). The organic phase was dried (MgSO₄) and concentrated. The residue was chromatographed on silicagel (6:1 heptane:EtOAc). The 2-phenyletylamine starting materials and the corresponding tetrahydroisoquinolones are listed in Table 3.

TABLE 3
Synthesis of methoxy-1,2,3,4-tetrahydroisoquinolines

| | 1,2,3,4-tetrahydro- | Yield (over 3 |
|---|---|---|
| Starting material | isoquinolines | steps) |
| 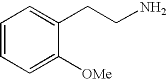 | 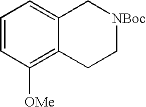 | 26% |
| 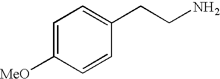 | 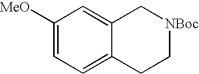 | 47% |
| 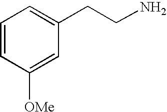 | 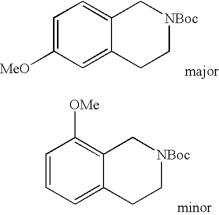 | 47% isomer ratio 5:1 |

### EXAMPLE 6

### Synthesis of dimethoxy-1,2,3,4-tetrahydroisoquinolines

6,7-Dimethoxy-1,2,3,4-tetrahydroisoquinoline and 5,6-dimethoxy-1,2,3,4-tetrahydroisoquinoline were synthesized as previously described (J. Med. Chem, 1994, (37), 1942-1954). By this procedure 6,7-dimethoxy-1,2,3,4-tetrahydroisoquinoline and 5,6-dimethoxy-1,2,3,4-tetrahydroisoquinoline were synthesized:

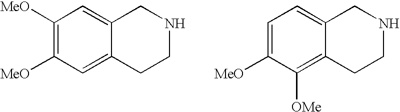

### EXAMPLE 7

### Chlorination of the Aromatic Ring in 1,2,3,4-tetrahydro-isoquinolines or Benzazepines

The starting material (1,2,3,4-tetrahydroisoquinoline or benzazepine;

1 eq.) was suspended in acetic acid (glacial) and SO₂Cl₂ (1.2 eq., 2.2 eq., or 3.0 eq., depending on the case) were added dropwise. After stirring for 2.5 hours the mixture was concentrated. Toluene was added and the mixture concentrated again. When needed to make purification easier the amine was Boc-protected, this was done by suspending the residue in THF or DMF. Di-tert-butyldicarbonate (1.2 eq.) and triethylamine (3 eq.) was added to the slurry. The mixture was stirred for 3 hours and then concentrated. The residue was dissolved in EtOAc and washed with Na₂CO₃ (sat.). The organic phase was dried (MgSO₄) and concentrated. The residue was chromatographed on silicagel (heptane:EtOAc). The tetrahydroisoquinoline or benzazepine starting materials and their chlorination products are listed in Table 4.

TABLE 4
Chlorination of 1,2,3,4-tetrahydro-isoquinolines
and benzazepines

| Starting | Equivalents | | Yield/Isomer |
|---|---|---|---|
| material | SO₂Cl₂ | Product | ratio |
| 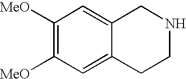 | 1.2 | 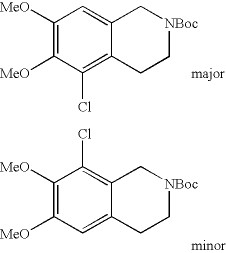 | 51% 1.7:1 |
| 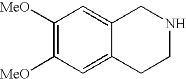 | 2.2 | 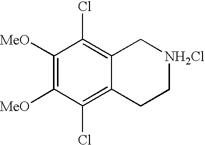 | 79% (no Boc) |
| 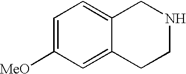 | 1.2 | 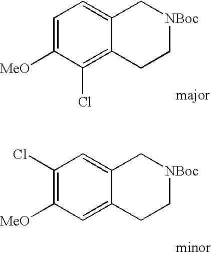 | 35% 5.5:1 |
| 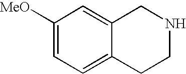 | 2.2 | 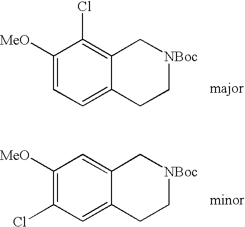 | 45% 3:1 |
| 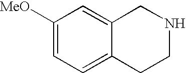 | 2.2 | 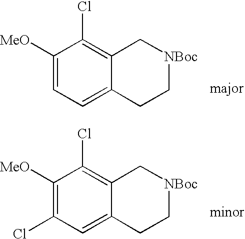 | 57% 2.2:1 |
| 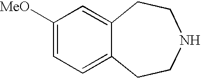 | 1.2 | 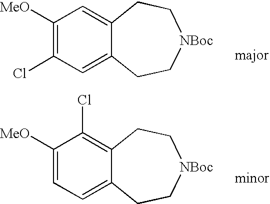 | 42% 2:1 |
| 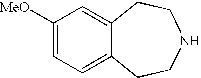 | 3.0 | 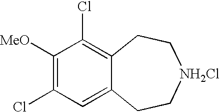 | Quantitative (no Boc) |
| 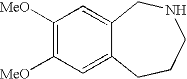 | 1.2 | 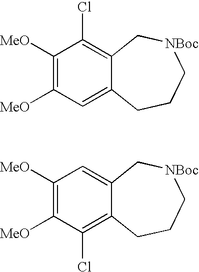 | 45% 1:1 |
| 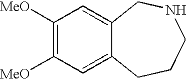 | 2.2 | 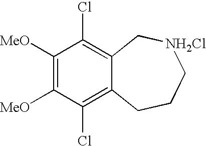 | Quantitative (no Boc) |
| 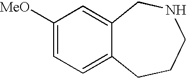 | 1.2 | 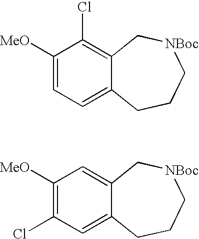 | 50% 1:1 |
| 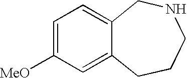 | 1.2 | 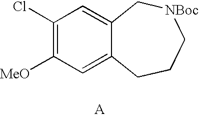 | 70% 4.5:2.2:1 A:B:C |
| | | 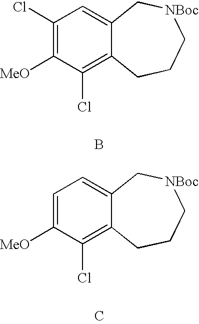 | |
| 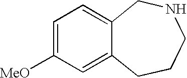 | 2.2 | 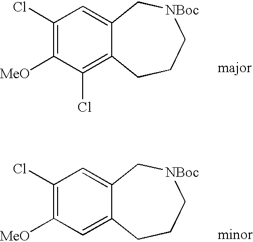 | 58% 11:1 |

### EXAMPLE 8

### Demethylation of Methylarylethers

The methylarylether (with or without the amine Boc-protected) was dissolved in concentrated hydrobromic acid. The mixture was heated to 105° C. for 3 hours and then concentrated. The residue was suspended in EtOAc and concentrated to afford the corresponding phenol as a gray solid. Yields were quantitative. The deprotected amines were coupled to isothiocyanates without further purification.

The demethylation of methoxy- and dimethoxyisoquinolines and of methoxy- and dimethoxy-tetrahydro-benzazepines is illustrated in Reaction Schemes D and E, respectively.

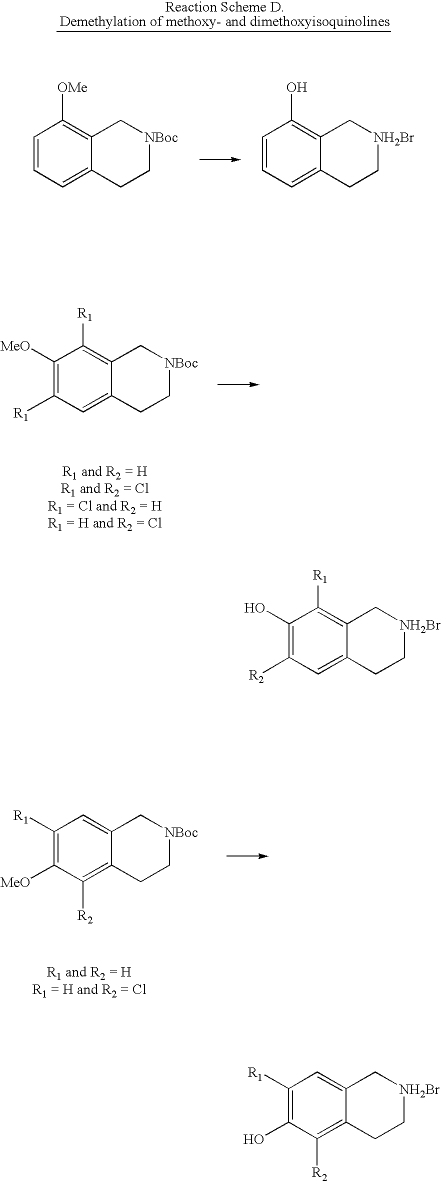

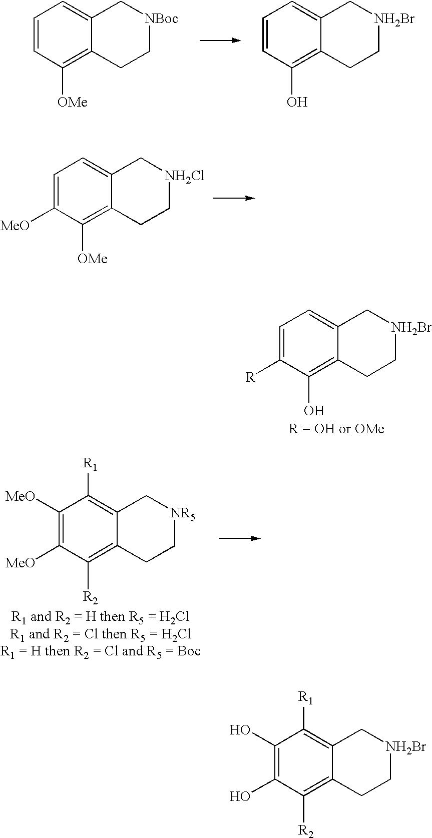

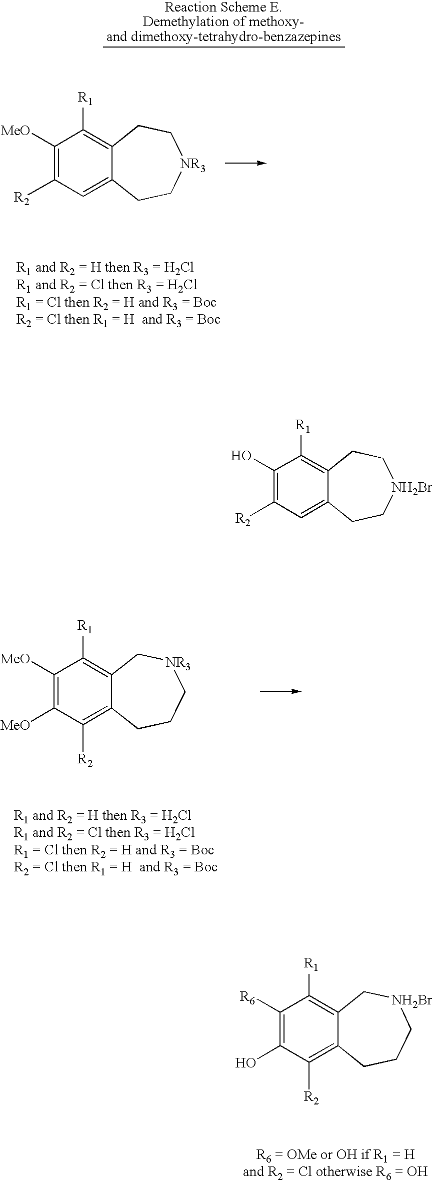

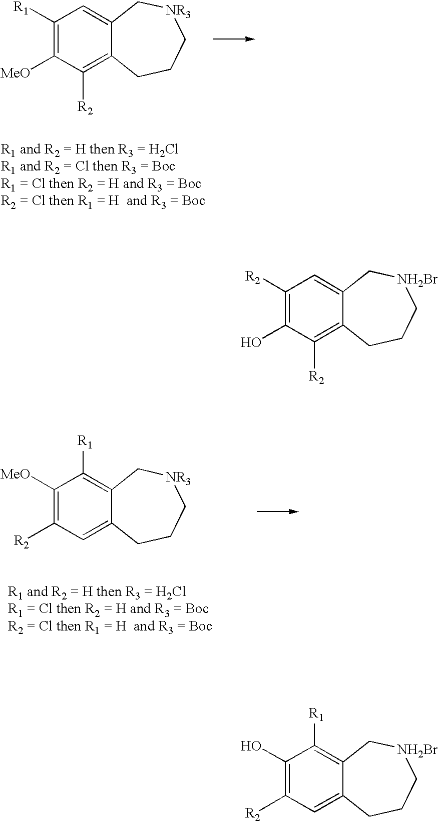

### EXAMPLE 9

### Synthesis of Isothiocyanates from Amines

Thiophosgene (CSCl₂, 1.1 eq.) was dissolved in EtOAc and stirred on ice. To this cold solution, a solution of the amine (1 eq.) and triethylamine in EtOAc was added drop wise. The mixture was allowed to reach room temperature. After 2.5 hours the mixture was diluted with EtOAc and washed with water. The organic phase was dried (MgSO₄) and concentrated. The remaining red-brown liquid was chromatographed on silicagel (heptane:EtOAc). The synthesis is illustrated by the Reaction Scheme F.

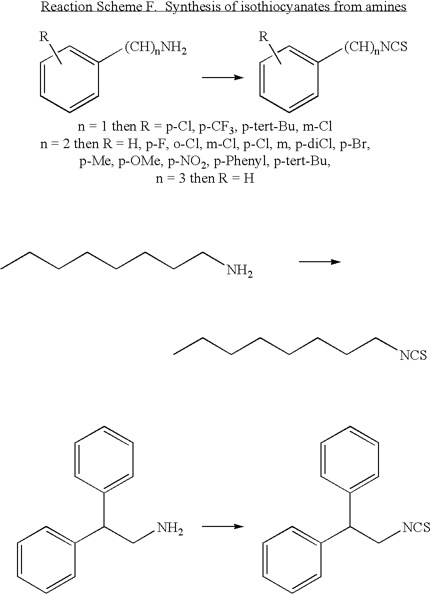

### EXAMPLE 10

### SYNTHESIS OF SUBSTITUTED THIOUREA COMPOUNDS OF THE INVENTION BY AMINE/ISOTHIOCYANATE COUPLING

The hydrobromic salt of the bicyclic amine (1 eq.) was dissolved in DMF and triethylamine (3 eq.) was added. This mixture was stirred for 15-30 minutes and then was the isothiocyanate (1.2 eq.) added. This mixture was stirred for 65 hours and then concentrated. The residue was dissolved in EtOAc and washed with water. The organic phase was dried (MgSO₄) and concentrated to give the crude product, typically as a yellow oil. The thiourea was chromatographed on silicagel (heptane:EtOAc). The substituted thioureas thus prepared are listed in Table 5.

TABLE 5
Substituted thioureas of the general formula
(I) obtained by amine/isothiocyanate coupling

| Name/ | | | |
|---|---|---|---|
| Code | Amine | Isothiocyanate | Substituted Thiourea |
| Capsaze- pine (prior art) | 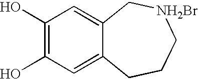 | 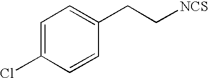 | 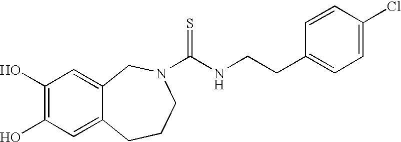 |
| Res-1-45 (prior art) | 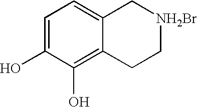 | 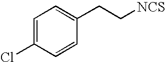 | 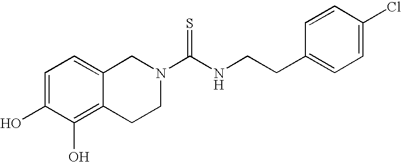 |
| Res-1-53 (prior art) | 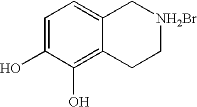 | 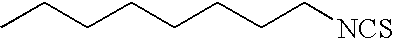 | 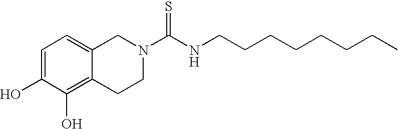 |
| Res-1-59 | 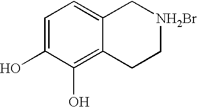 | 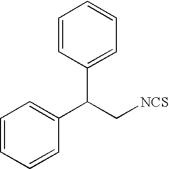 | 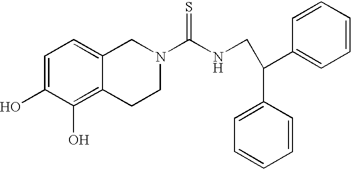 |
| Res-1-63 | 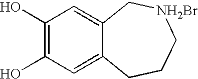 | 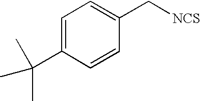 | 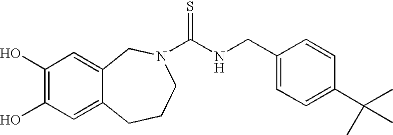 |
| Res-1-67 | 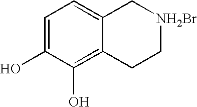 | 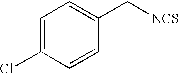 | 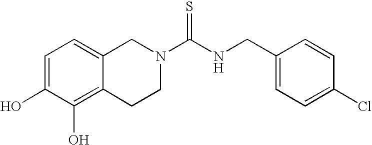 |
| Res-1-79 | 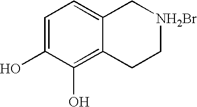 | 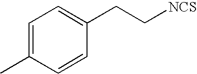 | 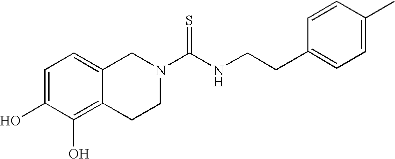 |
| Res-1-83 | 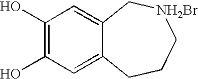 | 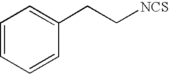 | 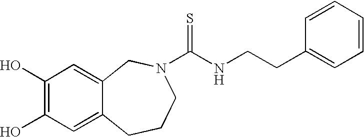 |
| Res-1-84 | 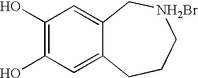 | 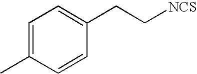 | 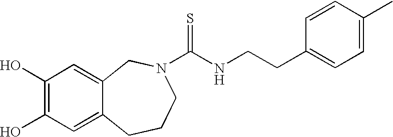 |
| Res-1-85 | 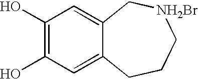 | 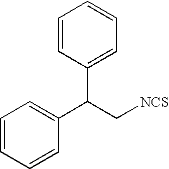 | 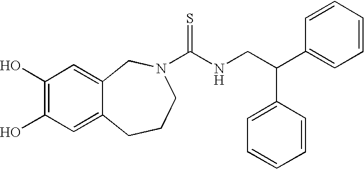 |
| Res-1-86 | 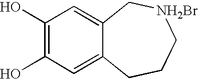 | 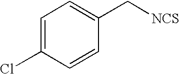 | 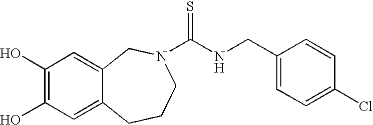 |
| Res-2-1 | 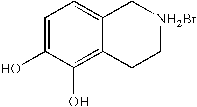 | 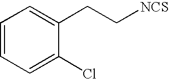 | 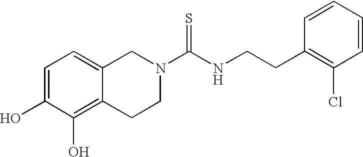 |
| Res-2-3 | 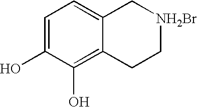 | 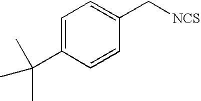 | 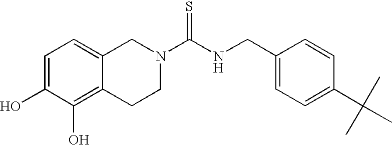 |
| Res-2-5 | 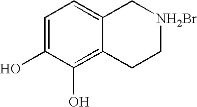 | 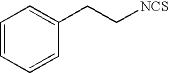 | 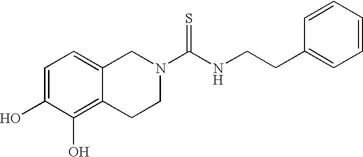 |
| Res-2- 5by | 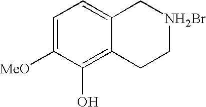 | 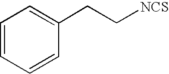 | 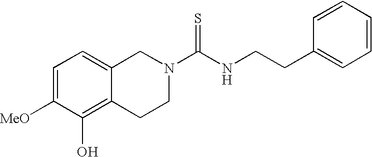 |
| Res-2-7 | 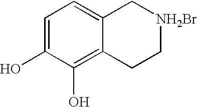 | 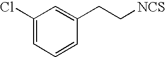 | 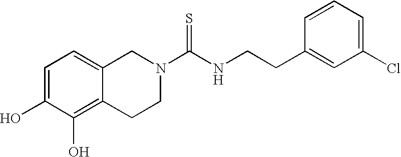 |
| Res-2-13 | 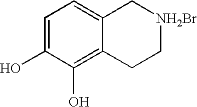 | 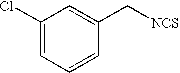 | 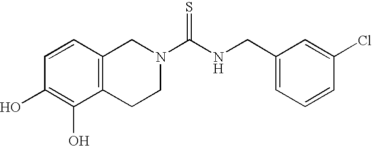 |
| Res-2-15 | 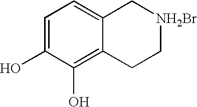 | 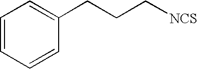 | 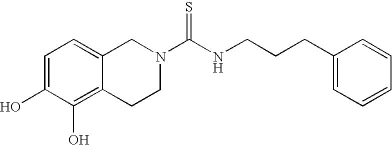 |
| Res-2-17 | 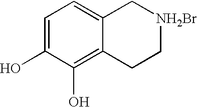 | 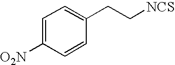 | 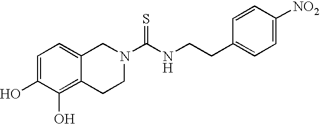 |
| Res-2-19 | 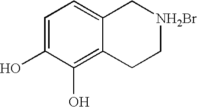 | 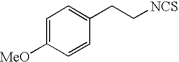 | 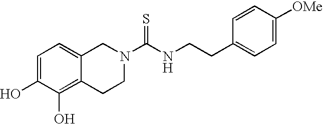 |
| Res-2- 29by | 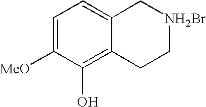 | 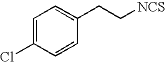 | 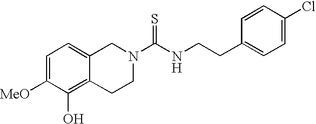 |
| Res-2-31 | 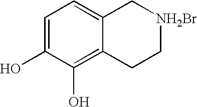 | 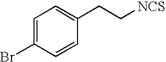 | 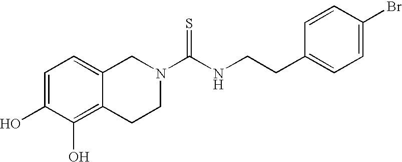 |
| Res-2- 31by | 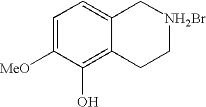 | 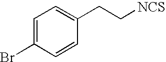 | 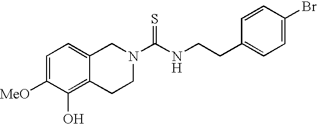 |
| Res-2-41 | 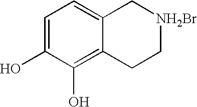 | 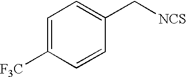 | 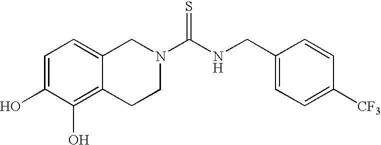 |
| Res-2-43 | 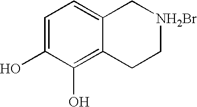 | 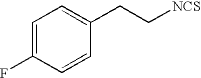 | 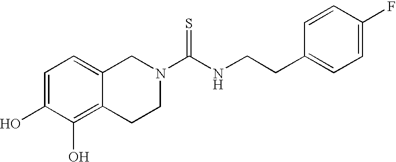 |
| Res-2- 43by | 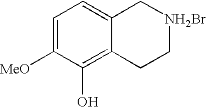 | 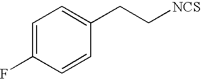 | 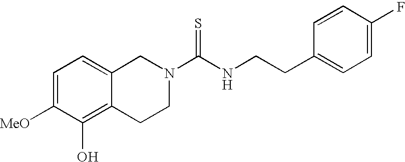 |
| Res-2-47 | 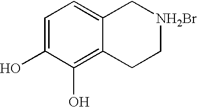 | 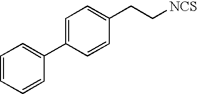 | 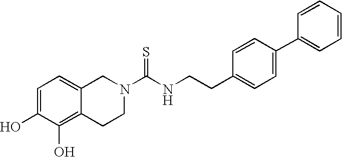 |
| Res-2- 47by | 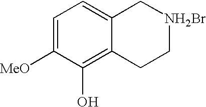 | 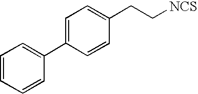 | 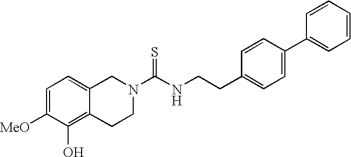 |
| Res-2-49 | 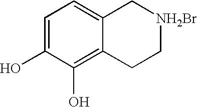 | 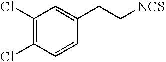 | 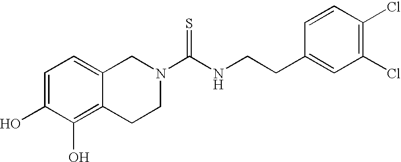 |
| Res-2-49 by | 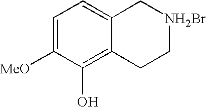 | 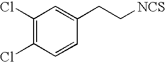 | 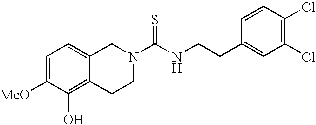 |
| Res-2-57 | 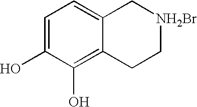 | 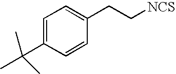 | 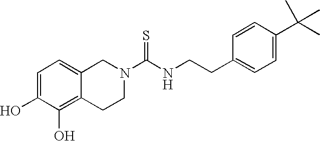 |
| Res-2-59 | 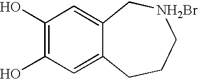 | 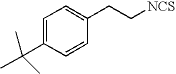 | 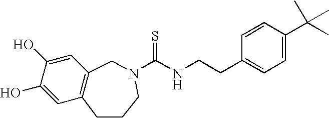 |
| Res-2-69 (prior art) | 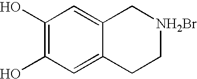 | 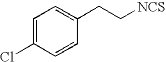 | 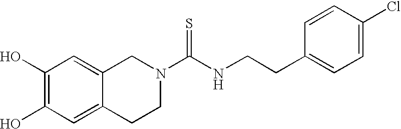 |
| Res-2-73 | 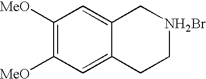 | 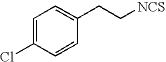 | 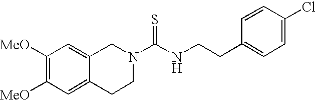 |
| Res-2-75 | 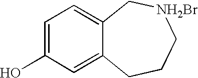 | 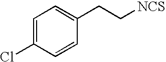 | 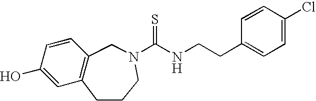 |
| Res-2-77 | 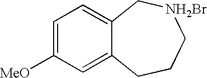 | 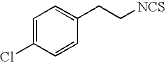 | 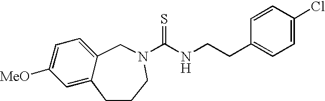 |
| Res-2-79 | 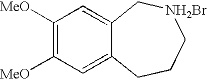 | 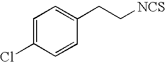 | 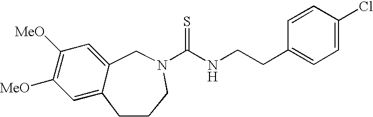 |
| Res-2-83 | 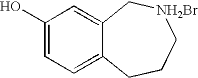 | 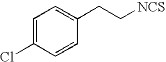 | 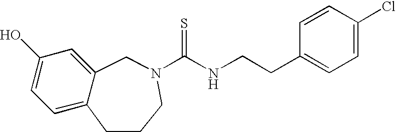 |
| Res-2-85 | 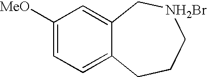 | 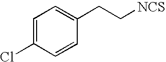 | 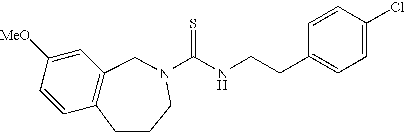 |
| Res-3-5 | 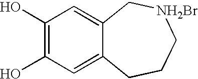 | 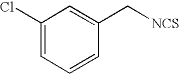 | 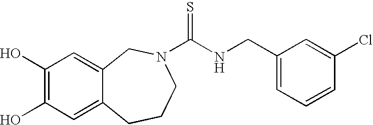 |
| Res-3-6 | 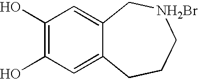 | 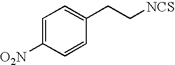 | 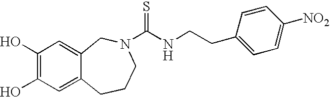 |
| Res-3-8 | 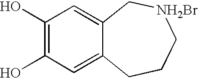 | 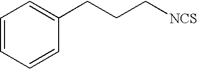 | 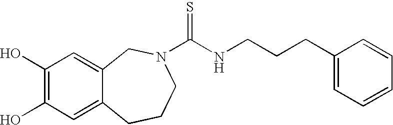 |
| Res-3-14 | 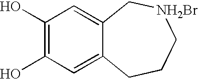 | 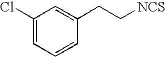 | 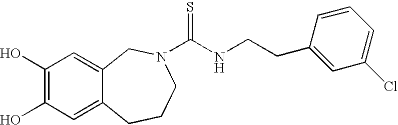 |
| Res-3-15 | 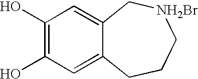 | 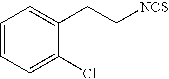 | 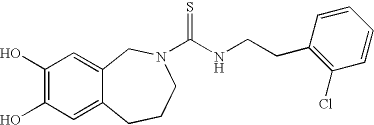 |
| Res-3-16 | 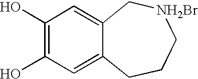 | 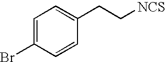 | 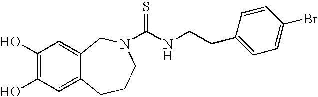 |
| Res-3-21 | 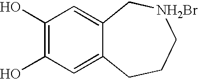 | 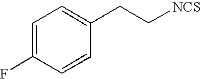 | 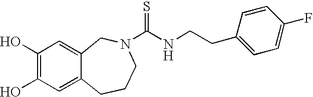 |
| Res-3-22 | 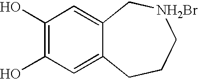 | 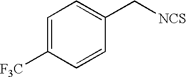 | 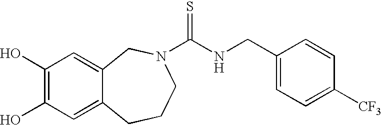 |
| Res-3-29 | 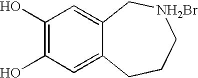 | 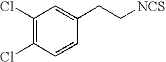 | 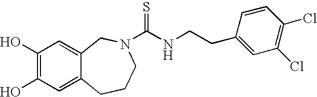 |
| Res-3-30 | 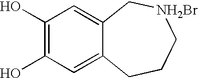 | 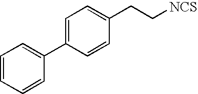 | 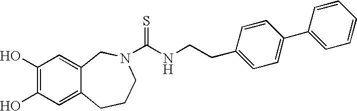 |
| Res-3-31 | 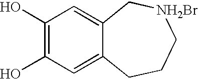 | 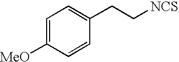 | 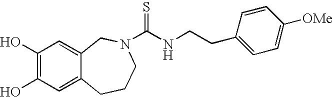 |
| Res-3-73 | 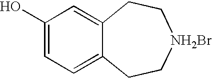 | 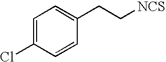 | 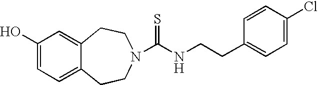 |
| Res-4-11 | 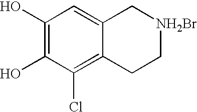 | 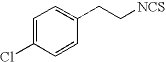 | 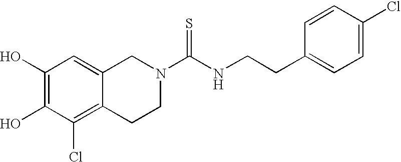 |
| Res-4-33 | 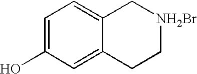 | 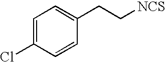 | 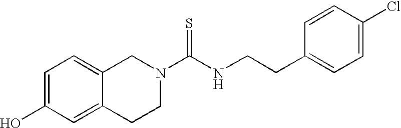 |
| Res-4-47 | 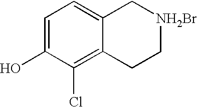 | 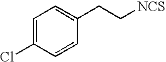 | 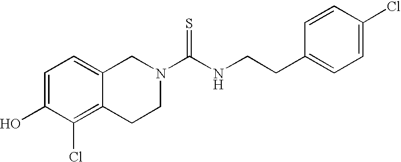 |
| Res-4-61 | 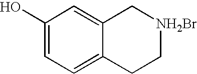 | 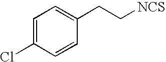 | 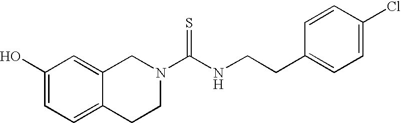 |
| Res-4-77- 1 | 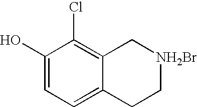 | 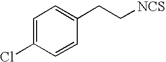 | 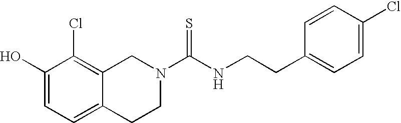 |
| Res-4-77- 2 | 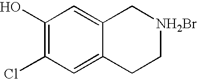 | 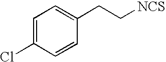 | 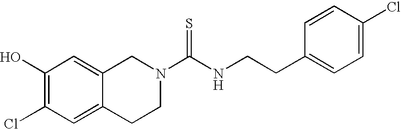 |
| Res-4-79 | 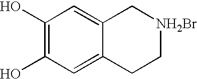 | 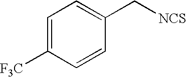 | 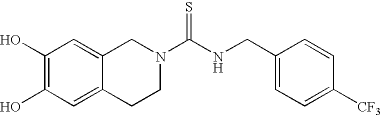 |
| Res-4-81 | 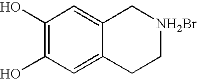 | 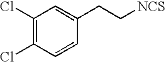 | 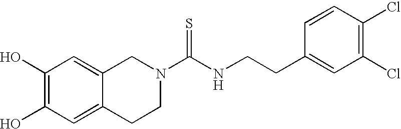 |
| Res-4-93 | 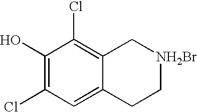 | 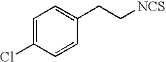 | 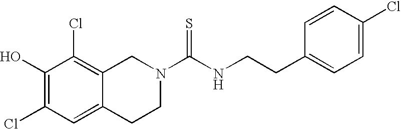 |
| Res-4-95 | 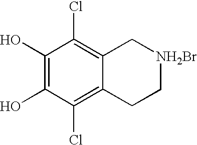 | 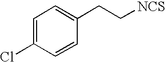 | 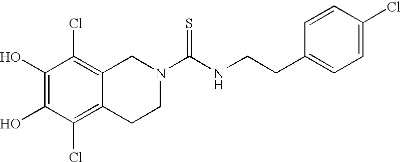 |
| Res-5-7 | 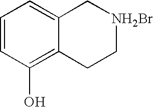 | 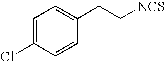 | 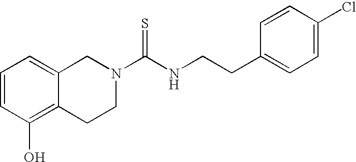 |
| Res-5-19 | 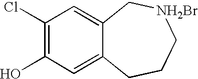 | 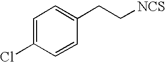 | 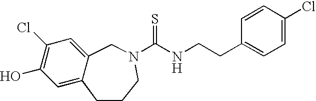 |
| Res-5-21 | 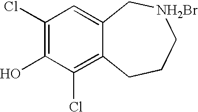 | 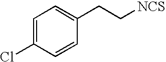 | 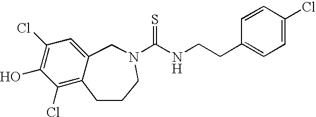 |
| Res-5-32 | 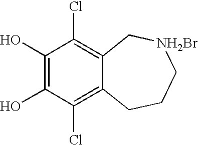 | 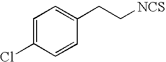 | 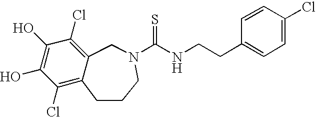 |
| Res-5-33A | 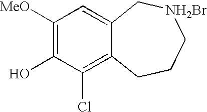 | 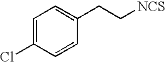 | 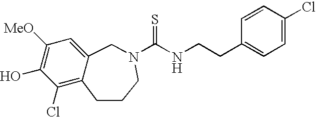 |
| Res-5-33B | 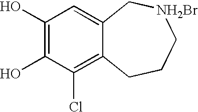 | 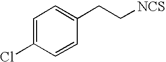 | 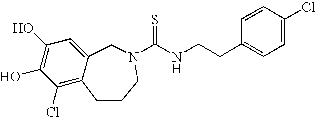 |
| Res-5-34 | 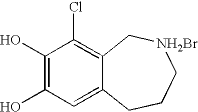 | 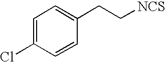 | 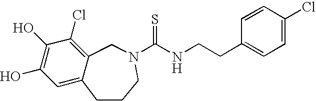 |
| Res-5-48B | 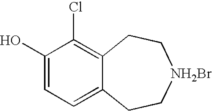 | 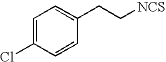 | 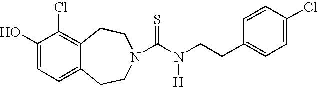 |
| Res-5-48C | 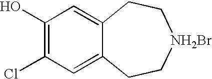 | 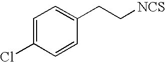 | 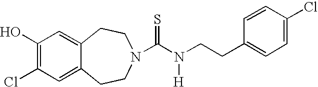 |
| Res-5-60B | 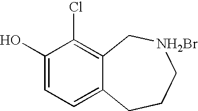 | 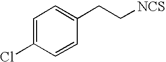 | 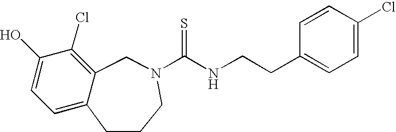 |
| Res-5-60C | 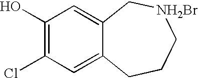 | 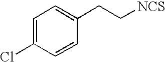 | 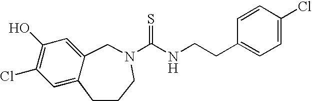 |
| Res-5-61 | 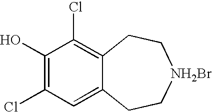 | 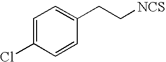 | 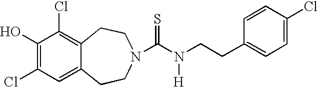 |
| Res-6-23 | 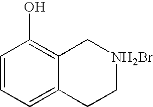 | 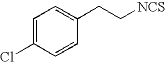 | 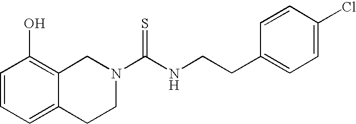 |
| Res-6-25 | 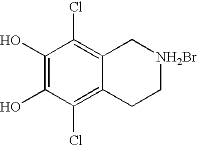 | 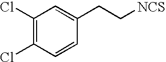 | 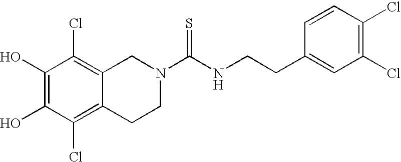 |
| Res-6-27 | 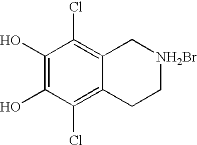 | 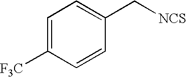 | 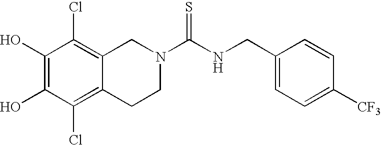 |

### B. SYNTHESIS OF SUBSTITUTED UREA COMPOUNDS OF THE INVENTION (D=O)

### EXAMPLE 11

### Synthesis of N-[2-(4-chlorophenyl)ethyl]-7,8-dihydroxy-1,3,4,5-tetrahydro-2H-2-benzazepine-2-carboxamide (Res 3-77)

The title compound was synthesized according to Scheme G.

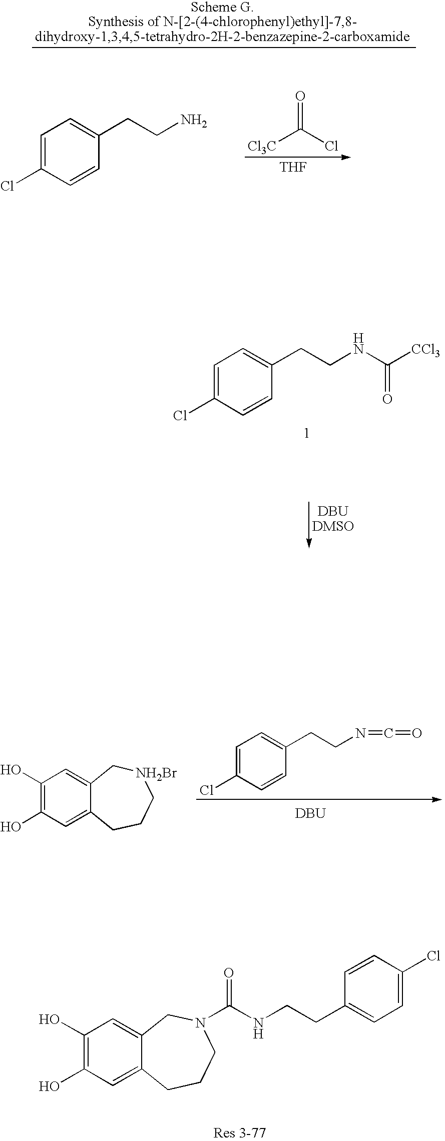

2,2,2-Trichloro-N-[2-(2-chlorophenyl)ethyl]acetamide. Trichloroacetyl chloride (1 eq.), was dissolved in THF (dry) under nitrogen, then 2-(4-chlorophenyl)ethyl amine (1 eq.) was added dropwise to the solution. The reaction mixture was stirred at room temperature for 3.5 hours. The mixture was concentrated and the residue chromatographed on silicagel (petroleum ether:EtOAc, 3:1) yielding 2,2,2-trichloro-N-[2-(2-chlorophenyl)ethyl]acetamide as white crystals (53%).

7,8-Dihydroxy-2,3,4,5-tetrahydro-1H-2-benzazepinium bromide salt was dissolved in DMSO (dry), DBU (1 eq.) was added and the solution stirred for 15 min. Then 2,2,2-trichloro-N-[2-(2-chlorophenyl)ethyl]acetamide and DBU (1 eq.) were added. The reaction mixture was stirred at 80° C. for 48 hours. CH₂Cl₂ was added to the solution and the organic phase was washed with HCl (3% in H₂O) and NaHCO₃ (sat.). The organic phase was dried (MgSO₄) and concentrated. The residue was chromatographed on silicagel (2% MeOH in CH₂Cl₂).

### EXAMPLE 12

### 2-[4-(4-Chlorophenyl)butanoyl]-2,3,4,5-tetrahydro-1H-2-benzazepine-7,8-diol (Res 3-85)

The title compound was synthesized according to Reaction Scheme H.

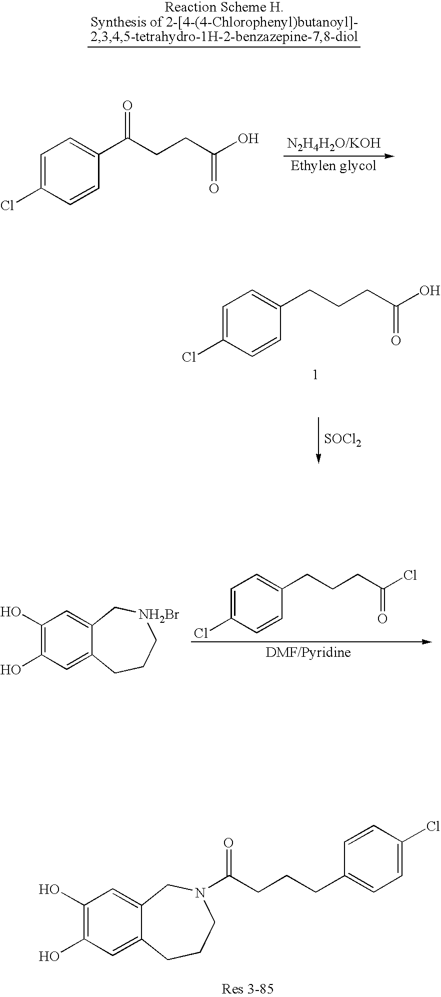

4-(4-Chlorophenyl)butanoic acid. (1) A mixture of 4-(4-chlorophenyl)4-oxobutanoic acid (1 eq.), KOH (3 eq.) and hydrazine hydrate (2.2 eq.) in ethylene glycol was refluxed azeotropically at 120-130° C. for 5 hours, the temperature was increased gradually to 180° C. Heating under reflux was then continued at 190° C. for 3 hours. The reaction mixture was cooled to 25° C., diluted with water and poured into a solution 2.5N HCl to give white crystals of 4-(4-chlorophenyl)butanoic acid (89%).

Solution A. 4-(4.chlorophenyl)butanoic acid (1.6 eq.) was dissolved in SOCl₂ and refluxed under nitrogen for 4 hours. Then the remaining SOCl₂ was evaporated and the residue dissolved in DMF (dry).

Solution B. 7,8-dihydroxy-2,3,4,5-tetrahydro-1H-2-benzazepinium bromide (1 eq.) was dissolved in DMF (dry), pyridine (1 eq) was added, and the solution stirred for 30 minutes at room temperature.

Solution A was then poured into solution B and pyridine (9 eq.) were added. The reaction mixture was stirred under nitrogen at room temperature for 24 hours. Then the mixture was concentrated and the residue chromatographed silicagel (gradient elution, 0-5% MeOH in CH₂Cl₂).

### EXAMPLE 13

### YIELDS AND PHYSICAL DATA OF THE COMPOUNDS OF THE INVENTION

General. ¹H-NMR spectra and ¹³C-NMR spectra were recorded with either of the following spectrometers: Bruker 300-DRX (at 300/75 MHz), Bruker DRX400 (at 400/100 MHz) or Bruker ARX-500 (500/125 MHz). CD₃OD (3.31/49.0 ppm), CDCl₃ (7.26/77.2 ppm) and (CD₃)₂SO (2.50/39.5 ppm) were used as solvents for NMR (calibration value shown in parenthesis). ESI-MS spectra were recorded on a MicroMass Q-TOF Micro spectrometer. All compounds were obtained as oils.

Res-1-45. N-[2-(4-chlorophenyl)ethyl]-5,6-dihydroxy-3,4-dihydroisoquinoline-2(1H)-carbothioamide. Yield: 44%. Physical data as previously reported (J. Med. Chem, 1994, 37, 1942-1954).

Res-1-53. 5,6-dihydroxy-N-octyl-3,4-dihydroisoquinoline-2(1H)-carbothioamide. Yield: 33%. Physical data as previously reported (J. Med. Chem, 1994, 37, 1942-1954).

Res-2-69. N-[2-(4-chlorophenyl)ethyl]-6,7-dihydroxy-3,4-dihydroisoquinoline-2(1H)-carbothioamide. Yield: 73%. Physical data as previously reported (J. Med. Chem, 1994, 37, 1942-1954).

Res-1-59. N-(2,2-diphenylethyl)-5,6-dihydroxy-3,4-dihydroisoquinoline-2(1H)-carbothioamide. Yield: 47%. ¹H-NMR (CD₃OD 400 MHz) ι 2.75 (t, J=6.0 Hz, 2H), 3.78 (t, J=6.0 Hz, 2H), 4.22 (d, J=8.1 Hz, 2H), 4.62 (s, 2H), 4.69 (t, J=8.1 Hz, 1H), 6.40 (d, J=8.2 Hz, 1H), 6.63 (d, J=8.2 Hz, 1H), 7.19 (m, 2H), 7.28 (m, 8H). ¹³C-NMR (CD₃OD 100 MHz) δ 23.6, 46.5, 50.3, 50.8, 51.1, 114.2, 118.0, 123.6, 126.2, 127.5, 127.5, 129.4, 129.4, 129.4, 129.4, 129.5, 129.5, 129.5, 129.5, 143.4, 143.8, 143.8, 144.6, 181.8. ESI-MS calculated for C₂₄H₂₅N₂O₂S (M+H) 405.1656, found 405.1636.

Res-1-63. N-(4-tert-butylbenzyl)-7,8-dihydroxy-1,3,4,5-tetrahydro-2H-2-benzazepine-2-carbothioamide. Yield: 42%. ¹H-NMR (CD₃OD 400 MHz) δ 1.28 (s, 9H), 1.82 (m, 2H), 2.80 (m, 2H), 4.12 (bs, 2H), 4.72 (s, 2H), 4.79 (s, 2H), 4.79 (s, 2H), 6.62 (s, 1H), 6.80 (s, 1H), 7.09 (d, J=8.1 Hz, 2H), 7.29 (d, J=8.1 Hz, 2H). ¹³C-NMR (CD₃OD 100 MHz) δ 28.9, 31.8, 31.8, 31.8, 34.8, 35.2, 50.0, 54.9. 54.9, 118.2, 118.4, 126.2, 126.2, 126.4, 128.0, 128.0, 134.2, 137.3, 143.8, 145.3, 150.8, 181.6. ESI-MS calculated for C₂₂H₂₉N₂O₂S (M+H) 385.1949, found 385.1972.

Res-1-67. N-(4-chlorobenzyl)-5,6-dihydroxy-3,4-dihydroisoquinoline-2(1H)-carbothioamide. Yield: 36%. ¹H-NMR (CD₃OD 400 MHz) δ 2.87 (t, J=6.0 Hz, 2H), 3.98 (t, J=6.0 Hz, 2H), 4.85 (s, 2H), 4.90 (s, 2H), 6.52 (d, J=8.1 Hz, 1H), 6.67 (d, J=8.1 Hz, 1H), 7.29 (m, 4H). ¹³C-NMR (CD₃OD 100 MHz) δ 23.8, 46.9, 49.2, 50.5, 114.3, 118.1, 123.7, 126.3, 129.3, 129.3, 130.0, 130.0, 133.5, 139.7, 143.5, 144.7, 181.9. ESI-MS calculated for C₁₇H₁₈ClN₂O₂S (M+H) 349.0777, found 349.0808.

Res-1-79. 5,6-dihydroxy-N-[2-(4-methylphenyl)ethyl]-3,4-dihydroisoquinoline-2(1H)-carbothioamide. Yield: 33%. ¹H-NMR (CD₃OD 300 MHz) δ 2.28 (s, 3H), 2.83 (t, J=6.0 Hz, 2H), 2.89 (t, J=7.5 Hz, 2H), 3.81 (t, J=7.5 Hz, 2H), 3.91 (t, J=6.0 Hz, 2H), 4.75 (s, 2H), 6.49 (d, J=8.1 Hz, 1H), 6.66 (d, J=8.1 Hz, 1H), 7.08 (m, 4H). ¹³C-NMR (CD₃OD 75 MHz) δ 21.1, 23.7, 36.0, 46.6, 48.3, 50.2, 114.2, 118.0, 123.7, 126.3, 129.8, 129.8, 130.0, 130.0, 136.7, 137.6, 143.5, 144.7, 181.6. ESI-MS calculated for C₁₉H₂₃N₂O₂S (M+H) 343.1480, found 343.1471

Res-1-83. 7,8-dihydroxy-N-(2-phenylethyl)-1,3,4,5-tetrahydro-2H-2-benzazepine-2-carbothioamide. Yield: 58%. ¹H-NMR (CD₃OD 400 MHz) δ 1.76 (m, 2H), 2.77 (m, 2H), 2.87 (t, J=7.5 Hz, 2H), 3.76 (t, J=7.5 Hz, 2H), 4.03 (bs, 2H), 4.67 (s, 2H), 6.59 (s, 1H), 6.78 (s, 1H), 7.15 (m, 3H), 7.24 (m, 2H). ¹³C-NMR (CD₃OD 100 MHz) δ 28.8, 34.7, 36.4, 48.2, 54.2, 58.3, 118.2, 118.3, 127.2, 128.8, 129.4, 129.4, 129.9, 129.9, 134.1, 140.7, 143.8, 145.4, 181.2. ESI-MS calculated for C₁₉H₂₃N₂O₂S (M+H) 343.1480, found 343.1493.

Res-1-84. 7,8-dihydroxy-N-[2-(4methylphenyl)ethyl]-1,3,4,5-tetrahydro-2H-2-benzazepine-2-carbothioamide. Yield: 50%. ¹H-NMR (CD₃OD 400 MHz) δ 1.75 (m, 2H), 2.28 (s, 3H), 2.76 (m, 2H), 2.81 (t, J=7.5 Hz, 2H), 3.73 (t, J=7.5 Hz, 2H), 4.03 (bs, 2H), 4.66 (s, 2H), 6.59 (s, 1H), 6.76 (s, 1H), 7.04 (d, J=1.89 Hz, 4H). ¹³C-NMR (CD₃OD 100 MHz) δ 21.1, 28.8, 34.7, 35.9, 48.3, 54.9, 55.2, 118.2, 118.3, 129.1, 129.8, 129.8, 130.1, 130.1, 134.1, 136.8, 137.5, 143.8, 145.4, 181.1. ESI-MS calculated for C₂₀H₂₅N₂O₂S (M+H) 357.1636, found 385.1641.

Res-1-85. N-(2,2-diphenylethyl)-7,8-dihydroxy-1,3,4,5-tetrahydro-2H-2-benzazepine-2-carbothioamide. Yield: 88%. ¹H-NMR (CD₃OD 400 MHz) δ 1.61 (m, 2H), 2.63 (m, 2H), 3.84 (bs, 2H), 4.15 (d, J=8.1 Hz, 2H), 4.51 (bs, 2H), 4.57 (t, J=8.1 Hz, 1H), 6.54 (s, 1H), 6.57 (s, 1H), 7.22 (m, 10H). ¹³C-NMR (CD₃OD 100 MHz) δ 28.6, 34.5, 50.9, 51.1, 53.7, 55.5, 117.9, 118.2, 127.6, 127.7, 129,2, 129.3, 129.3, 129.3, 129.3, 129.5, 129.5, 129.5, 129.5, 129.6, 133.8, 143.7, 143.8, 145.3, 181,3. ESI-MS calculated for C₂₅H₂₇N₂O₂S (M+H) 419.1793, found 419.1789.

Res-1-86. N-(4-chlorobenzyl)-7,8-dihydroxy-1,3,4,5-tetrahydro-2H-2-benzazepine-2-carbothioamide. Yield: 63%. ¹H-NMR (CD₃OD 400 MHz) δ 1.82 (m, 2H), 2.80 (m, 2H), 4.12 (bs, 2H), 4.73 (s, 2H), 4.80 (s, 2H), 6.61 (s, 1H), 6.81 (s, 1H), 7.11 (d, J=8.4 Hz, 2H), 7.21 (d, J=8.4 Hz, 2H). ¹³C-NMR (CD₃OD 100 MHz) δ 28.8, 34.9, 49.3, 49.8, 55.0, 118.3, 118.5, 128.7, 129.3, 129.3, 129.8, 129.8, 133.4, 134.3, 139.4, 143.7, 145.3, 181.9. ESI-MS calculated for C₁₈H₂₀ClN₂O₂S (M+H) 363.0934, found 363.0906.

Res-2-1. N-[2-(2-chlorophenyl)ethyl]-5,6-dihydroxy-3,4-dihydroisoquinoline-2(1H)-carbothioamide. Yield: 32%. ¹H-NMR (CD₃OD 300 MHz) δ 2.84 (t, J=6.0 Hz, 2H), 3.11 (t, J=6.5 Hz, 2H), 3.88 (t, J=6.5 Hz, 2H), 3.982 (t, J=6.0 Hz, 2H), 4.76 (s, 2H), 6.48 (d, J=8.1 Hz, 1H), 6.66 (d, J=8.1 Hz, 1H), 7.18 (m, 2H), 7.27 (m, 1H), 7.35 (m, 1H). ¹³C-NMR (CD₃OD 75 MHz) δ 23.8, 34.0, 46.2, 46.7, 50.3, 114.3, 118.0, 123.7, 126.3, 128.0, 129.0, 130.4, 132.4, 135.1, 138.4, 143.5, 144.7, 181.8. ESI-MS calculated for C₁₈H₂₀ClN₂O₂S (M+H) 363.0934, found 363.0946.

Res-2-3. N-(4-tert-butylbenzyl)-5,6-dihydroxy-3,4-dihydroisoquinoline-2(1H)-carbothioamide. Yield: 19%. ¹H-NMR (CD₃OD 300 MHz) δ 1.30 (s, 9H), 2.87 (t, J=6.0 Hz, 2H), 3.98 (t, J=6.0 Hz, 2H), 4.84 (s, 2H), 4.88 (s, 2H), 6.51 (d, J=8.1 Hz, 1H), 6.66 (d, J=8.1 Hz, 1H), 7.25 (d, J=8.2 Hz, 2H), 7.34 (d, J=8.2 Hz, 2H). ¹³C-NMR (CD₃OD 75 MHz) δ 23.8, 31.8, 31.8, 31.8, 35.3, 46.9, 49.9, 50.5, 114.3, 118.1, 123.8, 126.2, 126.2, 126.3, 128.3, 128.3, 137.6, 143.5, 144.7, 150.9, 182.2. ESI-MS calculated for C₂₁H₂₆N₂NaO₂S (M+Na) 393.1613, found 393.1638.

Res-2-5. 5,6-dihydroxy-N-(2-phenylethyl)-3,4-dihydroisoquinoline-2(1H)-carbothioamide. Yield: 25%. ¹H-NMR (CD₃OD 300 MHz) δ 2.84 (t, J=6.0 Hz, 2H), 2.95 (t, J=7.5 Hz, 2H), 3.84 (t, J=7.5 Hz, 2H), 3.92 (t, J=6.0 Hz, 2H), 4.77(s, 2H), 6.50 (d, J=8.1 Hz, 1H), 6.67 (d, J=8.1 Hz, 1H), 7.24 (m, 5H). ¹³C-NMR (CD₃OD 75 MHz) δ 23.8, 36.5, 46.6, 48.3, 50.3, 114.3, 118.0, 123.7, 126.3, 127.2, 129.4, 129.4, 130.0, 130.0, 140.9, 143.5, 144.7, 181.7. ESI-MS calculated for C₁₈H₂₁N₂O₂S (M+H) 329.1323, found 329.1304.

Res-2-5by. 5-hydroxy-6-methoxy-N-(2-phenylethyl)-3,4-dihydroisoquinoline-2(1H)-carbothioamide. Yield: 23%. ¹H-NMR (CD₃OD 400 MHz) δ 2.85 (t, J=6.0 Hz, 2H), 2.95 (t, J=7.5 Hz, 2H), 3.85 (m, 2H), 3.85 (s, 3H), 3.93 (t, J=6.0 Hz, 2H), 4.81 (s, 2H), 6.61 (d, J=8.3 Hz, 1H), 6.81 (d, J=8.3 Hz, 1H), 7.24 (m, 5H). ¹³C-NMR (CD₃OD 100 MHz) δ 23.7, 36.5, 46.6, 48.3, 50.3, 56.5, 110.6, 117.6, 123.3, 127.2, 127.8, 129.4, 129.4, 129.9, 129.9, 138.5, 140.9, 147.4, 181.6. ESI-MS calculated for C₁₉H₂₃N₂O₂S (M+H) 343.1480, found 343.1461.

Res-2-7. N-[2-(3-chlorophenyl)ethyl]-5,6-dihydroxy-3,4-dihydroisoquinoline-2(1H)-carbothioamide. Yield: 61%. ¹H-NMR (CD₃OD 300 MHz) δ 2.84 (t, J=6.0 Hz, 2H), 2.94 (t, J=7.3 Hz, 2H), 3.83 (t, J=7.3 Hz, 2H), 3.91 (t, J=6.0 Hz, 2H), 4.76 (s, 2H), 6.49 (d, J=8.1 Hz, 1H), 6.66 (d, J=8.1 Hz, 1H), 7.20 (m, 4H). ¹³C-NMR (CD₃OD 75 MHz) δ 23.7, 36.0, 46.7, 47.8, 50.3, 114.3, 118.0, 123.7, 126.3, 127.3, 128.4, 130.0, 130.9, 135.1, 143.2, 143.5, 144.7, 181.7. ESI-MS calculated for C₁₈H₂₀ClN₂O₂S (M+H) 363.0934, found 363.0936.

Res-2-13. N-(3-chlorobenzyl)-5,6-dihydroxy-3,4-dihydroisoquinoline-2(1H)-carbothioamide. Yield: 33%. ¹H-NMR (CD₃OD 300 MHz) δ 2.87 (t, J=6.0 Hz, 2H), 3.98 (t, J=6.0 Hz, 2H), 4.84 (s, 2H), 4.90 (s, 2H), 6.51 (d, J=8.1 Hz, 1H), 6.67 (d, J=8.1 Hz, 1H), 7.24 (m, 4H). ¹³C-NMR (CD₃OD 75 MHz) δ 23.8, 47.0, 49.3, 50.6, 114.3, 118.1, 123.7, 126.2, 126.8, 127.8, 128.9, 130.8, 135.1, 143.3, 143.5, 144.7, 182.4. ESI-MS calculated for C₁₇H₁₈ClN₂O₂S (M+H) 349.0777, found 349.0787.

Res-2-15. 5,6-dihydroxy-N-(3-phenylpropyl)-3,4-dihydroisoquinoline-2(1H)-carbothioamide. Yield: 16%. ¹H-NMR (CD₃OD 300 MHz) δ 1.98 (m, 2H), 2.65 (t, J=7.4 Hz, 2H), 2.84 (t, J=6.0 Hz, 2H), 3.68 (t, J=7.4 Hz, 2H), 3.88 (t, J=6.0 Hz, 2H), 4.74 (s, 2H), 6.50 (d, J=8.1 Hz, 1H), 6.66 (d, J=8.1 Hz, 1H), 7.20 (m, 5H). ¹³C-NMR (CD₃OD 75 MHz) δ 23.8, 32.2, 34.4, 46.6, 46.7, 50.2, 114.3, 118.0, 123.7, 126.3, 126.8, 129.3, 129.3, 129.4, 129.4, 143.3, 143.4, 144.7, 181.6. ESI-MS calculated for C₁₉H₂₃N₂O₂S (M+H) 343.1480, found 343.1489.

Res-2-17. 5,6-dihydroxy-N-[2-(4-nitrophenyl)ethyl]-3,4-dihydroisoquinoline-2(1H)-carbothioamide. Yield: 17%. ¹H-NMR (CD₃OD 300 MHz) δ 2.84 (t, J=6.0 Hz, 2H), 3.09 (t, J=7.3 Hz, 2H), 3.90 (m, 4H), 4.75 (s, 2H), 6.47 (d, J=8.1 Hz, 1H), 6.66 (d, J=8.1 Hz, 1H), 7.45 (d, J=8.8 Hz, 2H), 8.12 (d, J=8.8 Hz, 2H). ¹³C-NMR (CD₃OD 75 MHz) δ 23.7, 36.2, 46.7, 47.3, 50.3, 114.2, 118.0, 123.7, 124.5, 124.5, 126.2, 131.1, 131.1, 143.5, 144.7, 147.9, 149.0, 181.8. ESI-MS calculated for C₁₈H₂₀N₃O₄S (M+H) 374.1174, found 374.1175.

Res-2-19. 5,6-dihydroxy-N-[2-(4-methoxyphenyl)ethyl]-3,4-dihydroisoquinoline-2(1H)-carbothioamide. Yield: 19%. ¹H-NMR (CD₃OD 300 MHz) δ 2.86 (m, 4H), 3.75 (s, 3H) 3.80 (m, 2H), 3.91 (d, J=6.0 Hz, 2H), 4.76 (s, 2H), 6.49 (d, J=8.1 Hz, 1H), 6.66 (d, J=8.1 Hz, 1H), 6.81 (d, J=8.7 Hz, 2H), 7.13 (d, J=8.7 Hz, 2H).

¹³C-NMR (CD₃OD 75 MHz) δ 23.7, 35.5, 46.6, 48.4, 50.2, 55.6, 114.2, 114.8, 114.8, 118.0, 123.7, 125.0, 126.3, 130.8, 130.8, 132.8, 144.7, 145.5, 181.6. ESI-MS calculated for C₁₉H₂₃N₂O₃S (M+H) 359.1429, found 359.1431.

Res-2-29by. N-[2-(4-chlorophenyl)ethyl]-5-hydroxy-6-methoxy-3,4-dihydroisoquinoline-2(1H)-carbothioamide. Yield: 17%. ¹H-NMR (CD₃OD 300 MHz) δ 2.85 (t, J=6.0 Hz, 2H), 2.94 (t, J=7.5 Hz, 2H), 3.80 (m, 2H), 3.85 (s, 3H), 3.93 (t, J=6.0 Hz, 2H), 4.80 (s, 2H), 6.60 (d, J=8.3 Hz, 1H), 6.81 (d, J=8.3 Hz, 1H), 7.22 (m, 4H). ¹³C-NMR (CD₃OD 75 MHz) δ 23.6, 35.7, 46.6, 47.9, 50.3, 56.5, 110.6, 117.7, 123.2, 127.7, 129.4, 129.4, 131.6, 131.6, 133.3, 139.7, 144.6, 147.3, 181.9. ESI-MS calculated for C₁₉H₂₂ClN₂O₂S (M+H) 377.1090, found 377.1076.

Res-2-31.N-[2-(4-bromophenyl)ethyl]-5,6-dihydroxy-3,4-dihydroisoquinoline-2(1H)-carbothioamide. Yield: 34%. ¹H-NMR (CD₃OD 300 MHz) δ 2.84 (t, J=6.0 Hz, 2H), 2.91 (t, J=7.4 Hz, 2H), 3.82 (t, J=7.4 Hz, 2H), 3.91 (t, J=6.0 Hz, 2H), 4.75 (s, 2H), 6.48 (d, J=8.1 Hz, 1H), 6.67 (d, J=8.1 Hz, 1H), 7.13 (d, J=8.3 Hz, 2H), 7.38 (d, J=8.3 Hz, 2H). ¹³C-NMR (CD₃OD 75 MHz) δ 23.7, 35.7, 46.6, 47.8, 50.3, 114.2, 118.0, 120.9, 123.7, 126.3, 131.9, 131.9, 132.4, 132.4, 140.1, 143.5, 144.7, 181.6. ESI-MS calculated for C₁₈H₂₀BrN₂O₂S (M+H) 407.0429, found 407.0435.

Res-2-31by. N-[2-(4-bromophenyl)ethyl]-5-hydroxy-6-methoxy-3,4-dihydroisoquinoline-2(1H)-carbothioamide. Yield: 15%. ¹H-NMR (CD₃OD 400 MHz) δ 2.88 (t, J=6.0 Hz, 2H), 2.92 (t, J=7.6 Hz, 2H), 3.83 (t, J=7.6 Hz, 2H), 3.85 (s, 3H), 3.91 (t, J=6.0 Hz, 2H), 4.79 (s, 2H), 6.62 (d, J=8.2 Hz, 1H), 6.78 (d, J=8.2 Hz, 1H), 7.13 (d, J=8.4 Hz, 2H), 7.38 (d, J=8.4 Hz, 2H). ¹³C-NMR (CD₃OD 100 MHz) δ 23.3, 35.5, 46.2, 47.5, 49.9, 56.4, 110.3, 117.5, 120.6, 122.9, 127.3, 131.5, 131.5, 132.1, 132.1, 139.4, 144.0, 146.9, 181.3. ESI-MS calculated for C₁₉H₂₁BrN₂NaO₂S (M+Na) 443.0405, found 443.0436.

Res-2-41. 5,6dihydroxy-N-[4-(trifluoromethyl)benzyl]-3,4-dihydroisoquinoline-2(1H)-carbothioamide. Yield: 22%. ¹H-NMR (CD₃OD 400 MHz) δ 2.89 (t, J=6.0 Hz, 2H), 4.00 (t, J=6.0 Hz, 2H), 4.87 (s, 2H), 4.99 (s, 2H), 6.52 (d, J=8.1 Hz, 1H), 6.67 (d, J=8.1 Hz, 1H), 7.49 (d, J=8.1 Hz, 2H), 7.58 (d, J=8.1 Hz, 2H). ¹³C-NMR (CD₃OD 100 MHz) δ 23.8, 47.0, 49.4, 50.6, 114.3, 118.1, 123.7, 125.8 (q, J=202 Hz), 126.1 (q, J=4 Hz), 126.1 (q, J=4 Hz), 126.3, 128.8, 128.8, 129.9 (q, J=24 Hz), 143.5, 144.8, 145.6, 182.6. ESI-MS calculated for C₁₈H₁₈F₃N₂O₂S (M+H) 383.1072, found 383.1041.

Res-2-43. N-[2-(4-fluorophenyl)ethyl]-5,6-dihydroxy-3,4-dihydroisoquinoline-2(1H)-carbothioamide. Yield: 22%. ¹H-NMR (CD₃OD 300 MHz) δ 2.84 (t, J=6.0 Hz, 2H), 2.92 (t, J=7.5 Hz, 2H), 3.81 (t, J=7.5 Hz, 2H), 3.91 (t, J=6.0 Hz, 2H), 4.76 (s, 2H), 6.49 (d, J=8.1 Hz, 1H), 6.67 (d, J=8.1 Hz, 1H), 6.97 (m, 2H), 7.21 (m, 2H). ¹³C-NMR (CD₃OD 75 MHz) δ 23.7, 35.6, 46.6, 48.2, 50.3, 114.2, 115.9 (d, J=21 Hz), 115.9 (d, J=21 Hz), 118.0, 123.7, 126.3, 131.5 (d, J=10 Hz), 131.5 (d, J=10 Hz), 136.7 (d, J=3 Hz), 143.5, 144.7, 162.9 (d, J=241 Hz), 181.6. ESI-MS calculated for C₁₈H₂₀FN₂O₂S (M+H) 347.1229, found 347.1221.

Res-2-43by. N-[2-(4-fluorophenyl)ethyl]-5-hydroxy-6-methoxy-3,4-dihydroisoquinoline-2(1H)-carbothioamide. Yield: 9%. ¹H-NMR (CD₃OD 400 MHz) δ 2.86 (t, J=6.0 Hz, 2H), 2.94 (t, J=7.5 Hz, 2H), 3.82 (t, J=7.5 Hz, 2H), 3.86 (s, 3H), 3.94 (t, J=6.0 Hz, 2H), 4.81 (s, 2H), 6.62 (d, J=8.3 Hz, 1H), 6.82 (d, J=8.3 Hz, 1H), 6.99 (m, 2H), 7.23 (m, 2H). ¹³C-NMR (CD₃OD 100 MHz) δ 23.7, 35.6, 46.6, 48.2, 50.3, 56.5, 110.7, 115.9 (d, J=21 Hz), 115.9 (d, J=21 Hz), 117.7, 123.3, 127.8, 131.6 (d, J_{F}=8 Hz), 131.6 (d, J_{F}=8 Hz), 136.8, 144.7, 147.4, 162.8 (d, J_{F}=241 Hz), 181.9. ESI-MS calculated for C₁₉H₂₂FN₂O₂S (M+H) 361.1386, found 361.1379.

Res-2-47. N-[2-(1,1′-biphenyl-4-yl)ethyl]-5,6-dihydroxy-3,4-dihydroisoquinoline-2(1H)-carbothioamide. Yield: 18%. ¹H-NMR (CD₃OD 300 MHz) δ 2.87 (t, J=5.9 Hz, 2H), 2.99 (t, J=7.5 Hz, 2H), 3.90 (m, 4H), 4.77 (s, 2H), 6.59 (d, J=8.1 Hz, 1H), 6.67 (d, J=8.1 Hz, 1H), 7.30 (m, 3H), 7.40 (m, 2H), 7.53 (m, 4H). ¹³C-NMR (CD₃OD 75 MHz) δ 23.4, 35.8, 46.6, 47.8, 49.9, 114.0, 117.9, 123.4, 125.9, 127.5, 127.5, 127.7, 127.7, 129.4, 129.4, 130.1, 130.1, 139.4, 140.0, 140.3, 141.8, 144.2, 154.0, 181.1. ESI-MS calculated for C₂₄H₂₄N₂O₂S (M+H) 405.1636, found 405.1645.

Res-2-47by. N-[2-(1,1′-biphenyl-4-yl)ethyl]-5-hydroxy-6-methoxy-3,4-dihydroisoquinoline-2(1H)-carbothioamide. Yield: 14%. ¹H-NMR (CD₃OD 400 MHz) δ 2.87 (t, J=6.0 Hz, 2H), 3.00 (t, J=7.4 Hz, 2H), 3.85 (s, 3H), 3.88 (t, J=7.4 Hz, 2H), 3.96 (t, J=6.0 Hz, 2H), 4.81 (s, 2H), 6.61 (d, J=8.3 Hz, 1H), 6.80 (d, J=8.3 Hz, 1H), 7.32 (m, 3H), 7.42 (t, J=7.8 Hz, 2H), 7.52 (d, J=8.2 Hz, 2H), 7.58 (d, J=7.8 Hz, 2H). ¹³C-NMR (CD₃OD 100 MHz) δ 23.7, 36.0, 46.6, 48.1, 50.3, 56.5, 110.7, 117.7, 123.3, 127.8, 127.9, 127.9, 128.0, 128.0, 128.1, 129.8, 129.8, 130.5, 130.5, 140.1, 140.5, 142.4, 144.6, 147.4, 181.9. ESI-MS calculated for C₂₅H₂₆N₂NaO₂S (M+Na) 441.1613, found 441.1619.

Res-2-49. N-[2-(3,4-dichlorophenyl)ethyl]-5,6-dihydroxy-3,4-dihydroisoquinoline-2(1H)-carbothioamide. Yield: 21%. ¹H-NMR (CD₃OD 400 MHz) δ 2.84 (t, J=6.0 Hz, 2H), 2.94 (t, J=7.4 Hz, 2H), 3.83 (t, J=7.4 Hz, 2H), 3.99 (t, J=6.0 Hz, 2H), 4.76 (s, 2H), 6.49 (d, J=8.1 Hz, 1H), 6.66 (d, J=8.1 Hz, 1H), 7.13 (dd, J=8.2, 1.9 Hz, 1H), 7.38 (d, J=8.2 Hz, 1H), 7.40 (d, J=1.9 Hz, 1H). ¹³C-NMR (CD₃OD 100 MHz) δ 23.8, 35.5, 46.7, 47.5, 50.3, 114.3, 118.0, 123.3, 126.3, 130.0, 131.0, 131.4, 132.0, 133.0, 141.8, 143.5, 144.7, 181.8. ESI-MS calculated for C₁₈H₁₈Cl₂N₂O₂S (M+H) 397.0544, found 397.0579.

Res-2-49by. N-[2-(3,4-dichlorophenyl)ethyl]-5-hydroxy-6-methoxy-3,4-dihydroisoquinoline-2(1H)-carbothioamide. Yield: 30%. ¹H-NMR (CD₃OD 400 MHz) δ 2.85 (t, J=6.0 Hz, 2H), 2.97 (t, J=7.0 Hz, 2H), 3.83 (t, J=7.0 Hz, 2H), 3.85 (s, 3H) 3.92 (t, J=6.0 Hz, 2H), 4.80 (s, 2H), 6.60 (d, J=8.3 Hz, 1H), 6.80 (d, J=8.3 Hz, 1H), 7.14 (d, J=8.2 Hz, 1H), 7.38 (d, J=8.2 Hz, 1H), 7.40 (s, 1H). ¹³C-NMR (CD₃OD 100 MHz) δ 23.7, 35.4, 46.6, 47.5, 50.3, 56.6, 110.7, 117.7, 123.2, 127.7, 130.0, 131.0, 131.4, 132.0, 133.1, 141.8, 144.6, 147.4, 181.9. ESI-MS calculated for C₁₉H₂₁Cl₂N₂O₂S (M+H) 411.0701, found 411.0718.

Res-2-57. N-[2-(4-tert-butylphenyl)ethyl]-5,6-dihydroxy-3,4-dihydroisoquinoline-2(1H)-carbothioamide. Yield: 12%. ¹H-NMR (CD₃OD 300 MHz) δ 1.29 (s, 9H), 2.84 (t, J=6.0 Hz, 2H), 2.91 (t, J=7.5 Hz, 2H), 3.82 (t, J=7.5 Hz, 2H), 3.93 (t, J=6.0 Hz, 2H), 4.75 (s, 2H), 6.49 (d, J=8.1 Hz, 1H), 6.67 (d, J=8.1 Hz, 1H), 7.14 (d, J=8.3 Hz, 2H), 7.30 (d, J=8.3 Hz, 2H). ¹³C-NMR (CD₃OD 75 MHz) δ 23.7, 31.8, 31.8, 31.8, 35.2, 35.9, 46.6, 48.3, 50.2, 114.2, 118.0, 123.7, 126.2, 126.3, 126.3, 129.6, 129.6, 137.8, 143.5, 144.7, 150.1, 181.6. ESI-MS calculated for C₂₂H₂₉N₂O₂S (M+H) 385.1949, found 385.1905.

Res-2-59. N-[2-(4-tert-butylphenyl)ethyl]-7,8-dihydroxy-1,3,4,5-tetrahydro-2H-2-benzazepine-2-carbothioamide. Yield: 72%. ¹H-NMR (CD₃OD 400 MHz) δ 1.28 (s, 9H), 1.72 (m, 2H), 2.74 (m, 2H), 2.83 (t, J=7.5 Hz, 2H), 3.74 (t, J=7.5 Hz, 2H), 4.00 (bs, 2H), 4.66 (s, 2H), 6.60 (s, 1H), 6.79 (s, 1H), 7.07 (d, J=8.3 Hz, 2H), 7.28 (d, J=8.3 Hz, 2H). ¹³C-NMR (CD₃OD 100 MHz) δ 28.8, 31.8, 31.8, 31.8, 34.7, 35.2, 35.8, 48.2, 54.5, 55.3, 118.2, 118.4, 126.3, 126.31, 128.5, 129.6, 129.6, 134.1, 137.6, 143.7, 145.3, 150.1, 181.1. ESI-MS calculated for C₂₃H₃₁N₂O₂S (M+H) 399.2107 found 399.2108.

Res-2-73. N-[2-(4-chlorophenyl)ethyl]-6,7-dimethoxy-3,4-dihydroisoquinoline-2(1H)-carbothioamide. Yield: 83%. ¹H-NMR (CD₃OD 3.31ppm) δ 2.83 (t, J=5.8 Hz, 2H), 2.95 (t, J=7.4 Hz, 2H), 3.82 (s, 3H), 3.82 (s, 3H), 3.84 (t, J=7.4 Hz, 2H), 3.96 (t, J=5.8 Hz, 2H), 4.79 (s, 2H), 6.73 (s, 1H), 6.79 (s, 1H), 7.23 (m, 4H).

¹³C-NMR (CD₃OD 100 MHz) δ 29.1, 35.7, 47.0, 47.9, 50.3, 56.5, 56.6, 111.0, 112.8, 126.6, 128.7, 129.4, 129.4, 131.6, 131.6, 133.0, 139.7, 149.2, 149.5, 182.1. ESI-MS calculated for C₂₀H₂₄ClN₂O₂S (M+H) 391.1247, found 391.1251.

Res-2-75. N-[2-(4-chlorophenyl)ethyl]-7-hydroxy-1,3,4,5-tetrahydro-2H-2-benzazepine-2-carbothioamide. Yield: 63%. ¹H-NMR (CD₃OD 400 MHz) δ 1.77 (m, 2H), 2.85 (m, 2H), 2.85 (t, J=7.0 Hz, 2H), 3.75 (t, J=7.0 Hz, 2H), 4.07 (bs, 2H), 4.70 (s, 2H), 6.50 (dd, J=8.1 Hz, J=2.5 Hz, 1H), 6.61, (d, J=2.5 Hz, 1H), 7.06 (d, J=8.1 Hz, 1H), 7.10 (d, J=8.4 Hz, 2H), 7.22 (d, J=8.4 Hz, 2H). ¹³C-NMR (CD₃OD 100 MHz) δ 28.6, 35.6, 36.7, 47.8, 49.6, 54.5, 113.1, 117.8, 128.5, 129.4, 129.4, 131.5, 131.5, 131.6, 132.9, 139.5, 144.3, 158.1, 181.2. ESI-MS calculated for C₁₉H₂₂ClN₂OS (M+H) 361.1141, found 361.1118.

Res-2-77. N-[2-(4-chlorophenyl)ethyl]-7-methoxy-1,3,4,5-tetrahydro-2H-2-benzazepine-2-carbothioamide. Yield: 87%. ¹H-NMR ((CD₃)₂SO 400 MHz) δ 1.70 (m, 2H), 2.80 (t, J=7.5 Hz, 2H), 2.89 (m, 2H), 3.61 (m, 2H), 3.72 (s, 3H), 4.04 (bs, 2H), 4.77 (s, 2H), 6.63 (dd, J=8.2 Hz, J=2.6 Hz, 1H), 6.76, (d, J=2.6 Hz, 1H), 7.18 (d, J=8.4 Hz, 2H), 7.29 (d, J=8.2 Hz, 1H), 7.31 (d, J=8.4 Hz, 2H), 7.45 (t, J=5.1 Hz, 1H). ¹³C-NMR ((CD₃)₂SO 100 MHz) δ 27.3, 34.0, 34.4, 46.5, 52.2, 53.4, 54.9, 109.9, 115.5, 128.21, 128.21, 129.2, 130.5, 130.5, 130.6, 130.7, 138.5, 143.2, 158.4, 179.4. ESI-MS calculated for C₂₀H₂₄ClN₂OS (M+H) 375.1298, found 375.1323.

Res-2-79. N-[2-(4-chlorophenyl)ethyl]-7,8-dimethoxy-1,3,4,5-tetrahydro-2H-2-benzazepine-2-carbothioamide. Yield: 26%. ¹H-NMR ((CD₃)₂SO 400 MHz) δ 1.69 (m, 2H), 2.78 (t, J=7.6 Hz, 2H), 2.85 (m, 2H), 3.61 (m, 2H), 3.70 (s, 3H), 3.72 (s, 3H) 4.07 (bs, 2H), 4.74 (s, 2H), 6.80 (s, 1H), 7.13 (s, 1H), 7.14 (d, J=8.4 Hz, 2H), 7.29 (d, J=8.4 Hz, 2H), 7.51 (t, J=5.1 Hz, 1H). ¹³C-NMR ((CD₃)₂ SO 100 MHz) δ 27.3, 33.7, 34.2, 46.6, 53.7, 54.6, 55.5, 55.7, 113.9, 114.4, 125.0, 128.2, 128.2, 130.4, 130.4, 130.6, 134.0, 138.5, 145.9, 162.3, 179.7. ESI-MS calculated for C₂₁H₂₆ClN₂O₂S (M+H) 405.1403, found 405.1426.

Res-2-83. N-[2-(4-chlorophenyl)ethyl]-8-hydroxy-1,3,4,5-tetrahydro-2H-2-benzazepine-2-carbothioamide. Yield: 62%. ¹H-NMR (CD₃OD 400 MHz) δ 1.74 (m, 2H), 2.83 (m, 2H), 2.85 (t, J=7.4 Hz, 2H) 3.75 (t, J=7.4 Hz, 2H), 4.02 (bs, 2H), 4.78 (s, 2H), 6.60 (dd, J=8.1 Hz, J=2.6 Hz, 1H), 6.82 (d, J=2.6 Hz, 1H), 6.96 (d, J=8.1 Hz, 1H), 7.10 (d, J=8.4 Hz, 2H), 7.19 (d, J=8.4 Hz, 2H). ¹³C-NMR (CD₃OD 100 MHz) δ 28.7, 34.6, 35.6, 47.9, 54.5, 55.7, 115.0, 118.0, 129.4, 129.4, 131.5, 131.5, 131.7, 132.9, 133.4, 138.6, 139.5, 156.5, 181.4. ESI-MS calculated for C₁₉H₂₂ClN₂OS (M+H) 361.1141, found 361.1155.

Res-2-85. N-[2-(4-chlorophenyl)ethyl]-8-methoxy-1,3,4,5-tetrahydro-2H-2-benzazepine-2-carbothioamide. Yield: 49%. ¹H-NMR (CD₃OD 400 MHz) δ 1.77 (m, 2H), 2.87 (m, 2H), 2.87 (t, J=7.2 Hz, 2H), 3.74 (s, 3H), 3.75 (t, J=7.2 Hz, 2H), 4.08 (bs, 2H), 4.80 (s, 2H), 6.72 (dd, J=8.3 Hz, J=2.7 Hz, 1.H), 6.92 (d, J=2.7 Hz, 1H), 7.07 (d, J=8.3 Hz, 1H), 7.08 (d, J=8.5 Hz, 2H), 7.18 (d, J=8.5 Hz). ¹³C-NMR (CD₃OD 100 MHz) δ 27.5, 33.5, 34.4, 46.6, 53.7, 54,4, 54.5, 112.1, 115.7, 128.2, 128.2 130.3, 130.3, 130.5, 131.7, 133.5, 137.5, 138.3, 158.1, 180.3. ESI-MS calculated for C₂₀H₂₄ClN₂OS (M+H) 375.1298, found 375.1334.

Res-3-5. N-(3-chlorobenzyl)-7,8-dihydroxy-1,3,4,5-tetrahydro-2H-2-benzazepine-2-carbothioamide. Yield: 40%. ¹H-NMR (CD₃OD 400 MHz) δ 1.83 (m, 2H), 2.81 (m, 2H), 4.13 (bs, 2H), 4.76 (s, 2H), 4.83 (s, 2H), 6.62 (s, 1H), 6.83 (s, 1H), 7.06 (d, J=7.0 Hz, 1H), 7.16 (d, J=7.0 Hz, 1H), 7.19 (m, 2H). ¹³C-NMR (CD₃OD 100 MHz) δ 28.9, 34.8, 49.2, 49.4, 55.0, 118.2, 118.5, 126.5, 127.7, 128.1, 128.7, 130.7, 134.2, 135.1, 143.2, 143.8, 145.4, 182.0. ESI-MS calculated for C₁₈H₂₀ClN₂O₂S (M+H) 363.0934, found 363.0952.

Res-3-6. 7,8-dihydroxy-N-[2-(4-nitrophenyl)ethyl]-1,3,4,5-tetrahydro-2H-2-benzazepine-2-carbothioamide. Yield: 45%. ¹H-NMR (CD₃OD 400 MHz) δ 1.72 (m, 2H), 2.76 (m, 2H), 3.00 (t, J=7.0 Hz, 2H), 3.83 (t, J=7.0 Hz, 2H), 4.03 (bs, 2H), 4.66 (s, 2H), 6.59 (s, 1H), 6.77 (s, 1H), 7.30 (d, J=8.3 Hz, 2H), 8.05 (d, J=8.3, 2H). ¹³C-NMR (CD₃OD 100 MHz) δ 28.8, 34.9, 36.2, 47.2, 54.7, 55.0, 118.2, 118.3, 124.4, 124.4, 128.8, 131.0, 131.0, 134.2, 143.7, 145.3, 147.9, 148.9, 181.3. ESI-MS calculated for C₁₉H₂₂N₃O₄S (M+H) 388.1331, found 388.1337.

Res-3-8. 7,8-dihydroxy-N-(3-phenylpropyl)-1,3,4,5-tetrahydro-2H-2-benzazepine-2-carbothioamide. Yield: 37%. ¹H-NMR (CD₃OD 400 MHz) δ 1.79 (m, 2H), 1.88 (dd, J=7.0 Hz, J=7.0 Hz, 2H), 2.55 (t, J=7.0 Hz, 2H), 2.79 (m, 2H), 3.60 (t, J=7.0 Hz, 2H), 4.08 (bs, 2H), 4.65 (s, 2H), 6.60 (s, 1H), 6.84 (s, 1H), 7.13 (m, 3H), 7.24 (m, 2H). ¹³C-NMR (CD₃OD 100 MHz) δ 28.9, 32.3, 34.2, 34.8, 46.6, 54.7, 54.7, 118.3, 118.3, 126.7, 128.8, 129.3, 129.3, 129.4, 129.4, 134.2, 143.3, 143.8, 145.4, 181.1. ESI-MS calculated for C₂₀H₂₅N₂O₂S (M+H) 357.1636, found 357.1641.

Res-3-14. N-[2-(3-chlorophenyl)ethyl]-7,8-dihydroxy-1,3,4,5-tetrahydro-2H-2-benzazepine-2-carbothioamide. Yield: 66%. ¹H-NMR (CD₃OD 400 MHz) δ 1.76 (m, 2H), 2.76 (m, 2H), 2.87 (t, J=7.3 Hz, 2H), 3.75 (t, J=7.3 Hz, 2H), 4.01 (bs, 2H), 4.68 (s, 2H), 6.59 (s, 1H), 6.79 (s, 1H), 7.05 (dd, J=7.1 Hz, J=1.7 Hz, 1H), 7.18 (m, 3H). ¹³C-NMR (CD₃OD 100 MHz) δ 28.8, 34.7, 36.0, 47.8, 54.3, 55.5, 118.2, 118.3, 127.3, 128.4, 128.6, 129.9, 130.9, 134.1, 135.1, 143.1, 143.7, 145.3, 181.2. ESI-MS calculated for C₁₉H₂₂ClN₂O₂S (M+H) 377.1090, found 377.1063.

Res-3-15. N-[2-(2-chlorophenyl)ethyl]-7,8-dihydroxy-1,3,4,5-tetrahydro-2H-2-benzazepine-2-carbothioamide. Yield: 22%. ^{—}H-NMR (CD₃OD 400 MHz) δ 1.75 (m, 2H), 2.77 (m, 2H), 3.15 (t, J=7.0 Hz, 2H), 3.80 (t, J=7.0 Hz, 2H), 4.02 (bs, 2H), 4.70 (s, 2H), 6.60 (s, 1H), 6.78 (s, 1H), 7.15 (m, 3H), 7.3 (m, 1H). ¹³C-NMR (CD₃OD 100 MHz) δ 28.8, 33.9, 34.7, 46.2, 54.1, 55.2, 118.2, 118.3, 128.1, 129.0, 130.0, 130.3, 132.5, 132.7, 134.1, 138.3, 143.8, 145.3, 181.4. ESI-MS calculated for C₁₉H₂₂ClN_{2l O}₂S (M+H) 377.1090, found 377.1046.

Res-3-16. N-[2-(4-bromophenyl)ethyl]-7,8dihydroxy-1,3,4,5-tetrahydro-2H-2-benzazepine-2-carbothioamide. Yield: 32%. ¹H-NMR (CD₃OD 400 MHz) δ 1.74 (m, 2H), 2.76 (m, 2H), 2.84 (t, J=7.3 Hz, 2H), 3.75 (t, J=7.3 Hz, 2H), 4.02 (bs, 2H), 4.69 (s, 2H), 6.60 (s, 1H), 6.81 (s, 1H), 7.05 (d, J=8.3 Hz, 2H), 7.38 ( d, J=8.3, 2H). ¹³C-NMR (CD₃OD 100 MHz) δ 28.8, 34.8, 35.8, 47.8, 54.5, 55.6, 118.2, 118.4, 120.9, 128.8, 131.9, 131.9, 132.4, 132.4, 134.1, 140.1, 143.7, 145.3, 181.2. ESI-MS calculated for C₁₉H₂₂BrN₂O₂S (M+H) 421.0585, found 421.0535.

Res-3-21. N-[2-(4-fluorophenyl)ethyl]-7,8-dihydroxy-1,3,4,5-tetrahydro-2H-2-benzazepine-2-carbothioamide. Yield: 26.4%. ¹H-NMR (CD₃OD 400 MHz) δ 1.75 (m, 2H), 2.77 (m, 2H), 2.85 (t, J=7.4 Hz, 2H), 3.75 (t, J=7.4 Hz, 2H), 4.03 (bs, 2H), 4.68 (s, 2H), 6.60 (s, 1H), 6.80 (s, 1H), 6.95 (m, 2H), 7.13 (m, 2H). ¹³C-NMR (CD₃OD 100 MHz) δ 28.8, 34.8, 35.5, 48.1, 54.3, 55.2, 115.9, 116.1, 118.2, 118.4, 128.8, 131.5, 131.6, 134.1, 136.6, 143.8, 154.4, 163.0 (d, J=2 Hz), 181.2. ESI-MS calculated for C₁₉H₂₂FN₂O₂S (M+H) 361.1386, found 361.1373.

Res-3-22. 7,8-dihydroxy-N-[4-(trifluoromethyl)benzyl]-1,3,4,5-tetrahydro-2H-2-benzazepine-2-carbothioamide. Yield: 24%. ¹H-NMR (CD₃OD 400 MHz) δ 1.84 (m, 2H), 2.83 (m, 2H), 4.15 (bs, 2H), 4.76 (s, 2H), 4.92 (s, 2H), 6.63 (s, 1H), 6.84 (s, 1H), 7.29 (d, J=8.0 Hz, 2H), 7.52 (d, J=8.0, 2H). ¹³C-NMR (CD₃OD 100 MHz) δ 28.9, 34.9, 49.49, 55.01, 55.01, 118.3, 118.6, 125.9 (q, J=275 Hz), 126.06 (q, J=4 Hz), 126.06 (q, J=4 Hz), 128.6, 128.6, 128.7, 130.3 (q, J=120 Hz), 134.3, 143.8, 145.4, 145.4, 182.2. ESI-MS calculated for C₁₉H₂₀F₃N₂O₂S (M+H) 397.1197, found 397.1193.

Res-3-29. N-[2-(3,4-dichlorophenyl)ethyl]-7,8-dihydroxy-1,3,4,5-tetrahydro-2H-2-benzazepine-2-carbothioamide. Yield: 38%. ¹H-NMR (CD₃OD 400 MHz) δ 1.75 (m, 2H), 2.77 (m, 2H), 2.88 (t, J=7.2 Hz, 2H), 3.76 (t, J=7.2 Hz, 2H), 4.01 (bs, 2H), 4.70 (s, 2H), 6.60 (s, 1H), 6.82 (s, 1H), 7.02 (dd, J=8.2 Hz, J=2.0 Hz 2H), 7.32 ( d, J=8.2, 1H), 7.34 (d, J=2.0 Hz 1H). ¹³C-NMR (CD₃OD 100 MHz) δ 28.8, 34.7, 35.4, 47.5, 54.1, 55.5, 118.2, 118.4, 128.8, 130.0, 130.9, 131.4, 132.0, 133.0, 134.1, 141.7, 143.7, 145.3, 181.3. ESI-MS calculated for C₁₉H₂₀Cl₂N₂O₂SNa (M+Na) 433.0521, found 433.0545

Res-3-30. N-[2-(1,1′-biphenyl-4-yl)ethyl]-7,8-dihydroxy-1,3,4,5-tetrahydro-2H-2-benzazepine-2-carbothioamide. Yield: 44%. ¹H-NMR (CD₃OD 400 MHz) δ 1.76 (m, 2H), 2.76 (m, 2H), 2.91 (t, J=7.3 Hz, 2H), 3.80 (t, J=7.3 Hz, 2H), 4.03 (bs, 2H), 4.70 (s, 2H), 6.60 (s, 1H), 6.82 (s, 1H), 7.23 (d, J=8.2 Hz, 2H), 7.29 (tt, J=7.3 Hz, J=1.2 Hz 1H), 7.42 ( 1, J=7.3, 2H), 7.50 (d, J=8.2 Hz 2H), 7,58 (dt, J=7.3 Hz, J=1.2 Hz 2H). ¹³C-NMR (CD₃OD 100 MHz) δ 28.8, 34.7, 36.0, 48.2, 54.2, 55.1, 118.2, 118.4, 127.9, 127.9, 128.0, 128.0, 128.1, 128.8, 129.8, 129.8, 130.4, 130.4, 134.1, 139.9, 140.4, 142.3, 143.8, 145.4, 181.2. ESI-MS calculated for C₂₅H₂₇N₂O₂S (M+H) 419.1793, found 419.1818.

Res-3-31. 7,8-dihydroxy-N-[2-(4-methoxyphenyl)ethyl]-1,3,4,5-tetrahydro-2H-2-benzazepine-2-carbothioamide. Yield: 48%. ¹H-NMR (CD₃OD 400 MHz) δ 1.75 (m, 2H), 2.77 (m, 2H), 2.79 (t, J=7.5 Hz, 2H), 3.72 (t, J=7.5 Hz, 2H), 3.75 (s, 3H), 4.03 (bs, 2H), 4.66 (s, 2H), 6.59 (s, 1H), 6.77 (s, 1H), 6.79 (d, J=8.3 Hz, 2H), 7.05 (d, J=8.3, 2H). ¹³C-NMR (CD₃OD 100 MHz) δ 28.8, 34.8, 35.5, 54.3, 55.1, 55.7, 58.3, 114.9, 114.9, 118.2, 118.3, 128.8, 130.8, 130.8, 132.7, 134.1, 143.8, 145.4, 159.6, 181.1. ESI-MS calculated for C₂₀H₂₅N₂O₃S (M+H) 373.1586, found 373.1554.

Res-3-73. N-[2-(4-chlorophenyl)ethyl]-7-hydroxy-1,2,4,5-tetrahydro-3H-3-benzazepine-3-carbothioamide. Yield: 72%. ¹H-NMR (CD₃OD 400 MHz) δ 2.83 (m, 4H), 2.92 (t, J=7.4 Hz, 2H), 3.81 (t, J=7.4 Hz, 2H), 3.89 (t, J=4.6 Hz, 2H), 3.95 (t, J=4.6 Hz, 2H), 6.54 (dd, J=8.1 Hz, J=2.5 Hz, 1H), 6.57 (d, J=2.5 Hz, 1H), 6.91 (d, J=8.1 Hz, 1H), 7.18 (d, J=8.5 Hz, 2H), 7.24 (d, J=8.5 Hz, 2H). ¹³C-NMR (CD₃OD 100 MHz) δ 35.7, 36.3, 37.4, 48.0, 51.5, 51.9, 113.9, 117.9, 129.4, 129.4, 131.6, 131.6, 132.0, 132.0, 133.0, 139.7, 142.4, 156.8, 181.6. ESI-MS calculated for C₁₉H₂₂ClN₂OS (M+H) 361.1141, found 361.1148.

Res 3-77. N-[2-(4-chlorophenyl)ethyl]-7,8-dihydroxy-1,3,4,5-tetrahydro-2H-2-benzazepine-2-carboxamide. Yield: 29% ¹H-NMR (CD₃OD 3.31 ppm): 1.46 (m, 2H), 2.50 (t, J=7.3 Hz, 2H), 2.60 (m, 2H), 3.12 (t, J=7.3 Hz, 2H), 3.40 (m, 2H), 4.11 (s, 2H), 6.43 (s, 1H), 6.54 (s, 1H), 6.83 (d, J=8.4 Hz, 2H), 6.99 (d, J=8.4 Hz, 2H) ¹³C-NMR (CD₃OD, 49.0 ppm) δ: 24.4, 34.3, 35.6, 41.9, 49.9, 51.2, 116.8, 117.1, 128.2, 128.2, 128.4, 130.3, 130.3, 131.7, 133.3, 138.5, 142.5, 143.8, 158.3 HRMS (ES+) calculated for C₁₉H₂₁ClN₂O₃ (M+) 360.1241, found 360.1241

Res 3-85. 2-[4-(4-Chlorophenyl)butanoyl]-2,3,4,5-tetrahydro-1H-2-benzazepine-7,8-diol Yield: 19%. ¹H-NMR (CDCl₃ 7.27 ppm): 1.74 (m, 2H), 1.91 (m,2H), 2.31 (t, J=7.4 Hz, 2H), 2.59 (t, J=7.4 Hz, 2H), 2.90 (m, 2H), 3.69 (bs, 2H), 4.48 (s, 2H), 6.71 (s, 1H), 7.03 (d, J=8.3 Hz, 2H), 7.17 (s, 1H), 7.20 (d, J=8.3 Hz, 2H). ¹³C-NMR (CDCl₃, 77.0 ppm) δ: 26.3, 29.6, 32.2, 34.4, 34.5, 51.0, 52.5, 116.0, 117.0, 128.4, 128.4, 129.1, 129.7, 129.7, 132.5, 132.8, 139.8, 142.0, 143.6, 172.5. ESI-MS calculated for C₂₀H₂₃ClN₂O₃ (M+H) 360.1366, found 360.1375.

Res-4-11. 5-chloro-N-[2-(4-chlorophenyl)ethyl]-6,7-dihydroxy-3,4-dihydroisoquinoline-2(1H)-carbothioamide. Yield: 24%. ¹H-NMR (CD₃OD 400 MHz) δ 2.81 (t, J=6.0 Hz, 2H), 2.93 (t, J=7.4 Hz, 2H), 3.82 (t, J=7.4 Hz, 2H), 3.95 (t, J=6.0 Hz, 2H), 4.77 (s, 2H), 6.55 (s, 1H), 7.23 (m, 4H). ¹³C-NMR (CD₃OD 100 MHz) δ 26.9, 35.6, 46.5, 47.9, 50.3, 112.2, 121.2, 125.0, 126.4, 129.4, 129.4, 131.5, 131.5, 133.0, 139.6, 142.1, 146.0, 182.0. ESI-MS calculated for C₁₈H₁₉Cl₂N₂O₂S (M+H) 397.0544, found 397.0585.

Res-4-33. N-[2-(4-chlorophenyl)ethyl]-6-hydroxy-3,4-dihydroisoquinoline-2(1H)-carbothioamide. Yield: 74%. 1H-NMR (CD₃OD 300 MHz) δ 2.82 (t, J=5.9 Hz, 2H), 2.92 (t, J=7.5 Hz, 2H), 3.83 (t, J=7.5 Hz, 2H), 3.89 (t, J=5.9 Hz, 2H), 4.73 (s, 2H), 6.64 (m, 2H), 6.95 (d, J=8.1 Hz, 1H), 7.19 (m, 4H).

¹³C-NMR (CD₃OD 75 MHz) δ 29.5, 35.3, 46.3, 47.4, 49.4, 114.3, 114.9, 124.7, 127.9, 129.0, 129.0, 130.8, 130.8, 132.5, 137.2, 138.6, 156.5, 181.0. ESI-MS calculated for C₁₈H₂₀ClN₂OS (M+H) 347.0985, found 347.0988.

Res-4-47. 5-chloro-N-[2-(4-chlorophenyl)ethyl]-6-hydroxy-3,4-dihydroisoquinoline-2(1H)-carbothioamide. Yield: 80%. ¹H-NMR (CD₃OD 300 MHz) δ 2.92 (t, J=5.9 Hz, 2H), 2.94 (t, J=7.6 Hz, 2H), 3.83 (t, J=7.6 Hz, 2H), 3.99 (t, J=5.9 Hz, 2H), 4.81 (s, 2H), 6.82 (d, J=8.3 Hz, 1H), 6.93 (d, J=8.3 Hz, 1H), 7.23 (m, 4H). ¹³C-NMR (CD₃OD 75 MHz) δ 27.6, 35.6, 46.2, 47.9, 50.2, 115.5, 121.7, 126.3, 127.1, 129.4, 129.4, 131.6, 131.6, 133.0, 135.2, 139.6, 153.2, 182.2. ESI-MS calculated for C₁₈H₁₉Cl₂N₂OS (M+H) 381.0595, found 381.0626.

Res-4-61. N-[2-(4-chlorophenyl)ethyl]-7-hydroxy-3,4-dihydroisoquinoline-2(1H)-carbothioamide. Yield: 22%. ¹H-NMR (CD₃OD 300 MHz) δ 2.80 (t, J=6.0 Hz, 2H), 2.93 (t, J=7.6 Hz, 2H), 3.84 (t, J=7.6 Hz, 2H), 3.89 (t, J=6.0 Hz, 2H), 4.80 (s, 2H), 6.61 (d, J=2.4 Hz, 1H), 6.66 (dd, J=8.2, 2.4 Hz, 1H), 6.99 (d, J=8.2 Hz, 1H), 7.21 (m, 4H). ¹³C-NMR (CD₃OD 75 MHz) δ 28.5, 35.3, 46.6, 47.5, 50.2, 113.3, 114.8, 126.7, 129.0, 129.0, 129.5, 130.9, 130.9, 132.6, 134.8, 138.6, 156.1, 181.1.

ESI-MS calculated for C₁₈H₂₀ClN₂OS (M+H) 347.0985, found 347.1000.

Res-4-77-1. 8-chloro-N-[2-(4-chlorophenyl)ethyl]-7-hydroxy-3,4-dihydroisoquinoline-2(1H)-carbothioamide. Yield: 53%. ¹H-NMR (CDCl₃ 300 MHz) δ 2.74 (t, J=5.7 Hz, 2H), 2.89 (t, J=7.1 Hz, 2H), 3.11, (bs, 2H), 3.85 (t, J=7.1 Hz, 2H), 3.93 (t, J=5.7 Hz, 2H), 4.66 (s, 2H), 6.76 (d, J=8.3 Hz, 1H), 6.86 (d, J=8.3 Hz, 1H), 7.11 (d, J=8.4 Hz, 2H), 7.20 (d, J=8.4 Hz, 2H). ¹³C-NMR (CDCl₃ 75 MHz) δ 27.9, 34.5, 45.7, 46.7, 47.4, 114.1, 117.9, 127.2, 127.5, 128.6, 128.6, 130.1, 130.1, 130.6, 132.2, 137.5, 150.8, 181.2. ESI-MS calculated for C₁₈H₁₉Cl₂N₂OS (M+H) 381.0595, found 381.0612.

Res-4-77-2. 6-chloro-N-[2-(4-chlorophenyl)ethyl]-7-hydroxy-3,4-dihydroisoquinoline-2(1H)-carbothioamide. Yield: 55%. ¹H-NMR (CDCl₃ 300 MHz) δ 2.77 (t, J=5.9 Hz, 2H), 2.84 (bs, 2H), 2.92 (t, J=7.2 Hz, 2H), 3.77 (t, J=7.2 Hz, 2H), 3.87 (t, J=5.9 Hz, 2H), 4.76 (s, 2H), 6.71 (s, 1H), 7.08 (d, 1H), 7.14 (d, J=8.4 Hz, 2H), 7.24 (d, J=8.4 Hz, 2H). ¹³C-NMR (CDCl₃ 75 MHz) δ 27.6, 34.6, 45.3, 46.7, 49.0, 114.0, 118.9, 127.3, 128.5, 128.6, 128.6, 130.1, 130.1, 132.2, 132.8, 137.5, 150.8, 180.9. ESI-MS calculated for C₁₈H₁₉Cl₂N₂OS (M+H) 381.0595, found 381.0616.

Res-4-79. 6,7-dihydroxy-N-[4-(trifluoromethyl)benzyl]-3,4-dihydroisoquinoline-2(1H)-carbothioamide. Yield: 54%. ¹H-NMR (CD₃OD 400 MHz) δ 2.79 (t, J=5.8 Hz, 2H), 4.00 (t, J=5.8 Hz, 2H), 4.82 (s, 2H), 5.01 (s, 2H), 6.60 (s, 1H), 6.63 (s, 1H), 7.51 (d, J=8.2 Hz, 2H), 7.61 (d, J=8.2 Hz, 2H). ¹³C-NMR (CD₃OD 100 MHz) δ 29.1, 47.5, 49.4, 50.4, 114.0, 115.7, 125.4, 126.0 (q, J=269 Hz), 126.1 (q, J=4 Hz), 126.1 (q, J=4 Hz), 127.6, 128.8, 128.8, 129.9 (q, J=32 Hz), 145.1, 145.5, 145.6, 182.7. ESI-MS calculated for C₁₈H₁₈F₃N₂O₂S (M+H) 383.1041, found 383.1076.

Res-4-81. N-[2-(3,4-dichlorophenyl)ethyl]-6,7-dihydroxy-3,4-dihydroisoquinoline-2(1H)-carbothioamide. Yield: 37%. ¹H-NMR (CD₃OD 300 MHz) δ 2.74 (t, J=5.9 Hz, 2H), 2.95 (t, J=7.4 Hz, 2H), 3.83 (t, J=7.4 Hz, 2H), 3.90 (t, J=5.9 Hz, 2H), 4.71 (s, 2H), 6.57 (s, 1H), 6.60 (s, 1H), 7.16 (dd, J=8.2 Hz, J=2.0 Hz, 1H), 7.40 (d, J=8.2 Hz, 1H), 7.41 (d, J=2.0 Hz, 1H). ¹³C-NMR (CD₃OD 75 MHz) δ 27.8, 34.3, 46.0, 46.4, 49.0, 112.7, 114.5, 124.2, 126.3, 128.8, 129.8, 130.2, 130.8, 131.9, 140.6, 143.9, 144.2, 180.7. ESI-MS calculated for C₁₈H₁₉Cl₂N₂O₂S (M+H) 397.0544, found 397.0533.

Res-4-93. 6,8-dichloro-N-[2-(4-chlorophenyl)ethyl]-7-hydroxy-3,4-dihydroisoquinoline-2(1H)-carbothioamide. Yield: 56%. ¹H-NMR (CD₃OD 400 MHz) δ 2.78 (t, J=5.7 Hz, 2H), 2.94 (t, J=7.4 Hz, 2H), 3.84 (t, J=7.4 Hz, 2H), 3.93 (t, J=5.7 Hz, 2H), 4.89 (s, 2H), 7.12 (s, 1H), 7.22 (m, 4H). ¹³C-NMR (CD₃OD 100 MHz) δ 28.6, 35.6, 46.1, 48.0, 49.5, 121.1, 121.5, 128.7, 129.3, 129.4, 129.4, 131.5, 131.5, 132.0, 133.0, 139.5, 139.6, 148.9. ESI-MS calculated for C₁₈H₁₈Cl₃N₂O₂S (M+H) 415.0205, found 415.0214.

Res-4-95. 5,8-dichloro-N-[2-(4-chlorophenyl)ethyl]-6,7-dihydroxy-3,4-dihydroisoquinoline-2(1H)-carbothioamide. Yield: 51%. ¹H-NMR (CD₃OD 400 MHz) δ 2.77 (t, J=5.8 Hz, 2H), 2.93 (t, J=7.4 Hz, 2H), 3.82 (t, J=7.4 Hz, 2H), 3.95 (t, J=5.8 Hz, 2H), 4.85 (s, 2H), 7.20 (m, 4H). ¹³C-NMR (CD₃OD 100 MHz) δ 27.1, 35.5, 45.8, 47.9, 49.3, 118.4, 120.2, 124.2, 125.8, 129.4, 129.4, 131.5, 131.5, 133.0, 139.5, 142.6, 142.9, 182.5. ESI-MS calculated for C₁₈H₁₈Cl₃N₂OS (M+H) 431.0154, found 431.0210.

Res-5-7. N-[2-(4-chlorophenyl)ethyl]-5-hydroxy-3,4-dihydroisoquinoline-2(1H)-carbothioamide. Yield: 65%. ¹H-NMR (CD₃OD 400 MHz) δ 2.81 (t, J=6.0 Hz, 2H), 2.94 (t, J=7.4 Hz, 2H), 3.83 (t, J=7.4 Hz, 2H), 3.96 (t, J=6.0 Hz, 2H), 4.84 (s, 2H), 6.62 (d, J=7.8 Hz, 1H), 6.67 (d, J=7.8 Hz, 1H), 7.01 (t, J=7.8 Hz, 1H), 7.23 (m, 4H). ¹³C-NMR (CD₃OD 100 MHz) δ 23.6, 35.7, 46.6, 47.9, 50.7, 113.8, 118.3, 123.1, 128.0, 129.4, 129.4, 131.5, 131.5, 133.0, 135.8, 139.6, 155.8, 182.0. ESI-MS calculated for C₁₈H₂₀ClN₂OS (M+H) 347.0985, found 347.1006.

Res-5-19. 8-chloro-N-[2-(4-chlorophenyl)ethyl]-7-hydroxy-1,3,4,5-tetrahydro-2H-2-benzazepine-2-carbothioamide. Yield: 38%. ¹H-NMR (CD₃OD 400 MHz) δ 1.75 (m, 2H), 2.84 (m, 4H), 3.75 (t, J=7.2 Hz, 2H), 4.02 (bs, 2H), 4.73 (s, 2H), 6.73 (s, 1H), 7.08 (d, J=8.1 Hz, 2H), 7.19 (d, J=8.1, 2H). 7.29 (s, 1H). ¹³C-NMR (CD₃OD, 100 MHz) δ 28.5, 35.3, 35.6, 47.8, 49.7, 54.5, 118.1, 119.0, 129.4, 129.4, 130.1, 131.5, 131.5, 132.0, 132.9, 139.4, 142.0, 153.4, 181.3. ESI-MS calculated for C₁₉H₂₁Cl₂N₂OS (M+H) 395.0751, found 395.0804.

Res-5-21. 6,8-dichloro-N-[2-(4-chlorophenyl)ethyl]-7-hydroxy-1,3,4,5-tetrahydro-2H-2-benzazepine-2-carbothioamide. Yield: 71%. ¹ H-NMR (CD₃OD 400 MHz) δ 1.78 (m, 2H), 2.85 (t, J=7.3 Hz, 2H) 3.13 (m, 2H), 3.75 (t, J=7.3 Hz, 2H), 3.97 (bs, 2H), 4.83 (s, 2H), 7.09 (d, J=8.5 Hz, 2H), 7.21 ( d, J=8.5, 2H), 7.33 (s, 1H). ¹³C-NMR (CD₃OD 100 MHz) δ 27.2, 30.6, 35.5, 47.8, 53.23, 54.68, 119.5, 123.5, 129.4, 129.4, 130.3, 131.0, 131.5, 131.5, 133.0, 139.5, 139.9, 150.0, 181.7.

ESI-MS calculated for C₁₉H₁₉Cl₃N₂OSNa (M+Na) 451.0182, found 451.0182.

Res-5-32. 6,9-dichloro-N-[2-(4-chlorophenyl)ethyl]-7,8-dihydroxy-1,3,4,5-tetrahydro-2H-2-benzazepine-2-carbothioamide. Yield: 44%. ¹H-NMR (CD₃OD 400 MHz) δ 1.82 (m, 2H), 2.88 (t, J=7.2 Hz, 2H), 3.06 (m, 2H), 3.82 (t, J=7.2 Hz, 2H), 4.07 (bs, 2H), 4.92 (s, 2H), 7.14 (d, J=8.4 Hz, 2H), 7.23 (d, J=8.4, 2H). ¹³C-NMR (CD₃OD 100 MHz) δ 27.2, 29.9, 35.5, 47.9, 51.1, 53.1, 120.2, 121.3, 126.3, 129.5, 129.5, 131.5, 131.5, 131.8, 133.1, 139.4, 142.1, 143.7, 181.7. ESI-MS calculated for C₁₉H₂₀Cl₃N₂O₂S (M+H) 445.0311, found 445.0313.

Res-5-33A. 6-chloro-N-[2-(4-chlorophenyl)ethyl]-7-hydroxy-8-methoxy-1,3,4,5-tetrahydro-2H-2-benzazepine-2-carbothioamid. Yield: 31%. ¹H-NMR (CD₃OD 500 MHz) δ 1.77 (m, 2H), 2.87 (t, J=7.3 Hz, 2H), 3.09 (m, 2H), 3.77 (t, J=7.3 Hz, 2H), 3.83 (s, 3H), 3.98 (bs, 2H), 4.83 (s, 2H), 6.97 (s, 1H), 7.06 (d, J=8.4 Hz, 2H), 7.20 (d, J=8.4, 2H). ¹³C-NMR (CD₃OD 125 MHz) δ 26.3, 28.5, 34.4, 46.6, 52.1, 54.3, 55.6, 112.0, 120.5, 128.2, 128.2, 128.2, 130.3, 130.3, 131.2, 131.8, 138.4, 142.3, 145.7, 180.3. ESI-MS calculated for C₂₀H₂₃Cl₂N₂O₂S (M+H) 425.0857, found 425.0874.

Res-5-33B. 6-chloro-N-[2-(4-chlorophenyl)ethyl]-7,8-dihydroxy-1,3,4,5-tetrahydro-2H-2-benzazepine-2-carbothioamide. Yield: 31%. ¹H-NMR (CD₃OD 500 MHz) δ 1.75 (m, 2H), 2.87 (t, J=7.3 Hz, 2H), 3.03 (m, 2H), 3.75 (t, J=7.3 Hz, 2H), 4.93 (bs, 2H), 4.77 (s, 2H), 6.82 (s, 1H), 7.01 (d, J=8.4 Hz, 2H), 7.21 ( d, J=8.4, 2H). ¹³C-NMR (CD₃OD 125 MHz) δ 27.6, 29.6, 35.6, 47.8, 52.8, 55.5, 116.8, 122.1, 129.4, 129.4, 129.6, 130.7, 131.6, 131.6, 133.0, 139.6, 142.3, 144.7, 181.4. ESI-MS calculated for C₁₉H₁₉Cl₂N₂O₂S (M-H) 409.0545, found 409.0557

Res-5-34. 9-chloro-N-[2-(4-chlorophenyl)ethyl]-7,8-dihydroxy-1,3,4,5-tetrahydro-2H-2-benzazepine-2-carbothioamide. Yield: 48%. ¹H-NMR (CD₃OD 400 MHz) δ 1.80 (m, 2H), 2.80 (m, 2H), 2.87 (t, J=7.0 Hz, 2H), 3.82 (t, J=7.0 Hz, 2H), 4.21 (bs, 2H), 4.80 (s, 2H), 6.60 (s, 1H), 7.13 (d, J=8.4 Hz, 2H), 7.22 (d, J=8.4, 2H).

¹³C-NMR (CD₃OD 100 MHz) δ 28.7, 35.5, 35.5, 47.9, 50.7, 55.4, 116.8, 121.1, 125.4, 129.5, 129.5, 131.5, 131.5, 133.1, 135.2, 139.4, 141,0, 146.6, 181.3. ESI-MS calculated for C₁₉H₂₁Cl₂N₂O₂S (M+H) 411.0701, found 411.0674.

Res-5-48B. 6-chloro-N-[2-(4-chlorophenyl)ethyl]-7-hydroxy-1,2,4,5-tetrahydro-3H-3-benzazepine-3-carbothioamide. Yield: 12%. ¹H-NMR (CD₃OD 400 MHz) δ 2.90 (t, J=7.3 Hz, 2H), 2.96 (t, J=5.5 Hz, 2H), 3.20 (t, J=5.5 Hz, 2H), 3.78 (t, J=7.3 Hz, 2H), 3.89 (t, J=5.5 Hz, 2H), 4.04 (t, J=5.5 Hz, 2H), 6.70 (d, J=8.2 Hz, 1H), 6.89 (d, J=8.2 Hz, 1H), 7.16 (d, J=8.4 Hz, 2H), 7.24 (d, J=8.4 Hz, 2H). ¹³C-NMR (CD₃OD 100 MHz) δ 32.6, 35.6, 35.8, 48.0, 49.7, 51.1, 114.8, 114.8, 129.5, 129.5, 129.9, 131.6, 131.6, 133.0, 133.1, 139.1, 139.7, 153.1, 182.2. ESI-MS calculated for C₁₉H₂₁Cl₂N₂OS (M+H) 395.0751, found 395.0769.

Res-5-48C. 7-chloro-N-[2-(4-chlorophenyl)ethyl]-8-hydroxy-1,2,4,5-tetrahydro-3H-3-benzazepine-3-carbothioamide. Yield: 28%. ¹H-NMR (CD₃OD 400 MHz) δ 2.82 (m, 4H), 2.92 (t, J=7.3 Hz, 2H), 3.80 (t, J=7.3 Hz, 2H), 3.89 (bs, 2H), 3.96 (bs, 2H), 6.69 (s, 1H), 7.04 (s, 1H), 7.16 (d, J=8.5 Hz, 2H), 7.24 (d, J=8.5 Hz, 2H). ¹³C-NMR (CD₃OD 100 MHz) δ 35.6, 36.0, 36.8, 48.0, 51.3, 51.6, 118.7, 119.3, 129.4, 129.4, 131.6, 131.6, 132.0, 133.0, 133.4, 139.7, 141.3, 152.4, 181.8. ESI-MS calculated for C₁₉H₂₁Cl₂N₂OS (M+H) 395.0751, found 395.0755

Res-5-60B. 9-chloro-N-[2-(4-chlorophenyl)ethyl]-8-hydroxy-1,3,4,5-tetrahydro-2H-2-benzazepine-2-carbothioamide. Yield: 23%. ¹H-NMR (CD₃OD 400 MHz) δ 2.82 (m, 2H), 2.86 (m, 4H), 3.81 (t, J=7.1 Hz, 2H), 4.19 (bs, 2H), 4.94 (s, 2H), 6.75 (d, J=8.2 Hz,1H), 6.94 (d, J=8.2 Hz, 1H), 7.12 (d, J=8.4 Hz, 2H), 7.21 (d, J=8.4 Hz, 2H). ¹³C-NMR (CD₃OD 100 MHz) δ 28.5, 35.0, 35.4, 47.9, 51.4, 54.9, 116.1, 120.8, 129.5, 129.5, 130.3, 131.5, 131.5, 133.1, 135.1, 135.6, 139.3, 152.8, 181.6. ESI-MS calculated for C₁₉H₂₁Cl₂N₂OS (M+H) 395.0751, found 395.0757.

Res-5-60C. 7-chloro-N-[2-(4-chlorophenyl)ethyl]-8-hydroxy-1,3,4,5-tetrahydro-2H-2-benzazepine-2-carbothioamide. Yield: 23%. ¹H-NMR (CD₃OD 400 MHz) δ 1.74 (m, 2H), 2.82 (m, 2H), 3.86 (t, J=7.4 Hz, 2H), 3.74 (t, J=7.4 Hz, 2H), 3.95 (bs, 2H), 4.83 (s, 2H), 6.98 (s, 1H), 7.08 (s, 1H), 7.10 (d, J=8.4 Hz, 2H), 7.20 (d, J=8.4 Hz, 2H). ¹³C-NMR (CD₃OD 100 MHz) δ 28.6, 34.5, 35.5, 47.8, 53.9, 55.6, 119.7, 119.9, 129.4, 129.4, 131.5, 131.6, 131.6, 132.9, 134.9, 137.9, 139.5, 151.9, 181.6. ESI-MS calculated for C₁₉H₂₁Cl₂N₂OS (M+H) 395.0765, found 395.0765

Res-5-61. 7,9-dichloro-N-[2-(4-chlorophenyl)ethyl]-8-hydroxy-1,3,4,5-tetrahydro-2H-2-benzazepine-2-carbothioamide. Yield: 42%. ¹H-NMR (CD₃OD 400 MHz) δ 2.89 (t, J=7.5 Hz, 2H), 2.95 (t, J=5.6 Hz, 2H), 3.17 (t, J=5.6 Hz, 2H), 3.77 (t, J=7.5 Hz, 2H), 3.86 (t, J=5.6 Hz, 2H), 4.40 (t, J=5.6 Hz, 2H), 7.06 (s, 1H), 7.16 (d, J=8.4 Hz, 2H), 7.23 (d, J=8.4 Hz, 2H). ¹³C-NMR (CD₃OD 100 MHz) δ 32.2, 35.6, 35.6, 48.0, 49.7, 50.7, 120.8, 123.8, 129.4, 129.4, 130.1, 131.5, 131.5, 133.0, 133.7, 137.9, 139.7, 149.1, 182.3. ESI-MS calculated for C₁₉H₁₉Cl₃N₂OSNa (M+Na) 451.0182, found 451.0228

Res-6-23. N-[2-(4-chlorophenyl)ethyl]-8-hydroxy-3,4-dihydroisoquinoline-2(1H)-carbothioamide. Yield: 55%. ¹H-NMR (CD₃OD 400 MHz) δ 2.74 (t, J=5.7 Hz, 2H), 2.85 (t, J=7.4 Hz, 2H), 3.75 (t, J=7.4 Hz, 2H), 3.94 (t, J=5.7 Hz, 2H), 4.63 (s, 2H), 6.55 (d, J=7.8 Hz, 1H), 6.56 (d, J=7.8 Hz, 1H), 6.92 (t, J=7.8 Hz, 1H), 7.14 (m, 4H). ¹³C-NMR (CD₃OD 100 MHz) δ 29.7, 35.8, 46.1, 47.0, 48.0, 113.2, 120.2, 120.9, 128.3, 129.4, 129.4, 131.6, 131.6, 133.0, 137.6, 139.7, 154.9, 182.3. ESI-MS calculated for C₁₈H₂₀ClN₂OS (M+H) 347.0985, found 347.0993.

Res-6-25. 5,8-dichloro-N-[2-(3,4-dichlorophenyl)ethyl]-6,7-dihydroxy-3,4-dihydroisoquinoline-2(1H)-carbothioamide. Yield: 49%. ¹H-NMR (CD₃OD 400 MHz) δ 2.71 (t, J=5.9 Hz, 2H), 2.86 (t, J=7.2 Hz, 2H), 3.75 (t, J=7.2 Hz, 2H), 3.88 (t, J=5.9 Hz, 2H), 4.76 (s, 2H), 7.05 (dd, J=8.2 Hz, J=2.0 Hz, 1H), 7.28 (d, J=8.2 Hz, 1H), 7.29 (d, J=2.0 Hz, 1H). ¹³C-NMR (CD₃OD 100 MHz) δ 27.1, 35.2, 45.8, 47.5, 49.4, 118.4, 120.3, 124.2, 125.9, 130.0, 131.0, 131.4, 132.0, 133.1, 141.8, 142.7, 143.0, 182.7. ESI-MS calculated for C₁₈H₁₇Cl₄N₂O₂S (M+H) 464.9765, found 464.9858.

Res-6-27. 5,8dichloro-6,7-dihydroxy-N-[4-(trifluoromethyl)benzyl]-3,4-dihydroisoquinoline-2(1H)-carbothioamide. Yield: 50%. ¹H-NMR (CD₃OD 400 MHz) δ 2.78 (t, J=6.0 Hz, 2H), 3.97 (t, J=6.0 Hz, 2H), 4.89 (s, 2H), 4.91 (s, 2H), 7.41 (d, J=8.1 Hz, 2H), 7.51 (d, J=8.1 Hz, 2H). ¹³C-NMR (CD₃OD 100 MHz) δ 27.3, 46.1, 49.6, 49.9, 118.5, 120.3, 125.8 (q, J=269 Hz), 125.9, 126.1 (q, J=4 Hz), 126.1 (q, J=4 Hz), 128.8, 128.8, 130.0 (q, J=32 Hz), 140.8, 142.7, 143.0, 145.5, 183.5. ESI-MS calculated for C₁₈H₁₆Cl₂N₂O₂S (M+H) 451.0261, found 451.0365.

### EXAMPLE 14

### Bronchorelaxation Test

Apparatus and materials

Dissection and mounting of lung tissue preparations. Lung tissue was obtained from patients undergoing lobectomia or pulmectomia due to lung carcinoma. The tissue was placed in a dissection chamber continuously perfused with 10 ml min⁻¹ of a physiological saline solution (PSS) at room temperature. An airway was identified in the cut part of the lobe, and a bronchus of 10-20 mm length and 1-2 mm diameter was obtained. The bronchus was cut into rings of a width of about 2-3 mm. Each bronchial ring was cleaved to obtain an about rectangular oblong preparation, one end of which was tied to a small steel hook connected to a force transducer, while the other end of the preparation was attached to a fixed hook. This is followed by a period of adjustment, as described below. The preparation was mounted in an atmosphere containing 12% of oxygen and 6% of CO₂.

Experimental chamber. The experimental chamber has a volume of 5 ml. It is perfused with PSS at a rate of 3 ml min⁻¹. Two preparations are mounted in the chamber, and measurements on them are performed in parallel. For mechanical tensioning each force transducer (AME 801, SensoNor A/S, Horten, Norway) is connected to a micrometer screw. The substances to be tested, the reference substance (capsazepine), and transmitter (LTD4) are injected upstream of the preparation(s).

Materials. PPS (physiological saline solution, in mM): NaCl, 117; KCl, 4.87; MgSO₄, 0.60; NaHCO₃, 25.0; CaCl₂, 1.60; glucose, 5.23. The solution is saturated with a mixture of 94% oxygen and 6% carbon dioxide, giving a pH of 7.40±0.05 in the experimental chamber. All substances are prepared as stock solution dissolved in the vehicles ethanol or DMSO. Leukotriene D4 (LTD4; Cayman Ltd.): 10 μl of a 100 μM ethanol stock solution. Capsazepine (Sigma Aldrich): 10 μl of a 0.1 M ethanol stock solution. Substance to be tested: 10-100 μl of a 0.01-0.1 M ethanol or DMSO stock solution. Solution for establishing the passive tension level: calcium-free PSS+2 mM EGTA+20 mM caffeine. To exclude effects by the test substance vehicle, ethanol or DMSO, respectively, were added during the entire experiment except during the presence of test substance.

Test Procedure

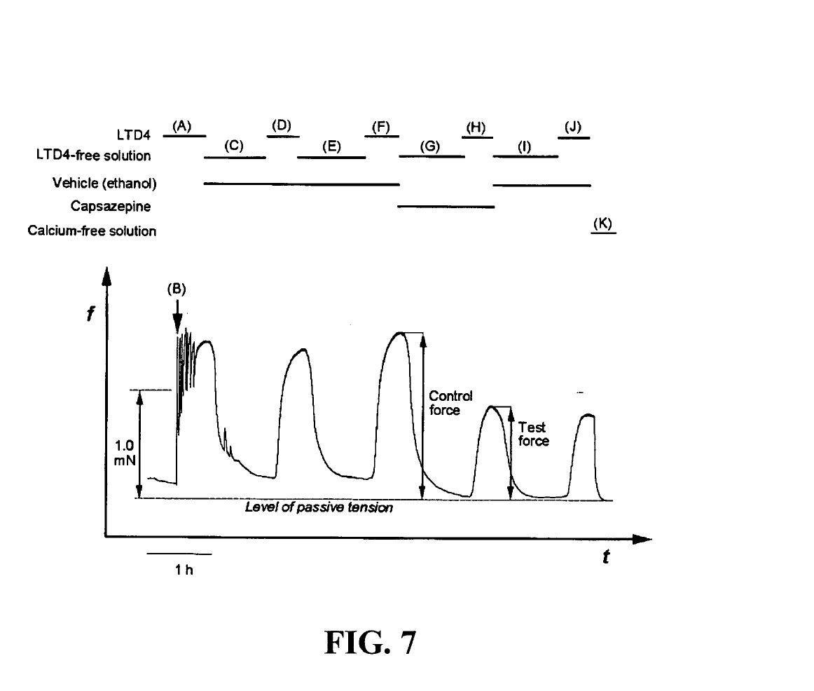

An exemplary test is shown in FIG. 7 in which capital letters indicate interference with the test system. The material for the preparation was a bronchus (inner diameter about 1 mm) from a male occasional smoker (41 yrs) but with the epithelium intact.

Adjustment and stretch. After mounting as described above the preparation is allowed to adjust with a low passive tone in the experimental chamber. The composition of the gas is changed to 94% (v/v) of oxygen. After a short adjustment period, PSS with 10 nM LTD4 is added to the experimental chamber upstream of the preparation (A). The preparation is stretched repeatedly (B) until it exerts a contraction force of around 150 mg. When the contraction has levelled off, leukotriene-free solution is administered for 1 hour (C), resulting in a relaxation. A second injection of 10 nM LTD4 (D) makes the preparation return to the tensioned state. At the peak tension leukotriene-free solution is again administered (E). After a third injection of 10 nM LTD4 (F) the preparation returns to the tensioned state. At the peak, PSS with 10 μM capsazepine (G) is added, resulting in a relaxation. After 1 h exposure to capsazepine, LTD4 is added, resulting in a contraction (H). In comparison with the control LTD4 contraction (F), a substantially weaker contraction is now observed (H). To obtain a measure of the test substance's bronchorelaxing effect the test and control forces registered in the experiment are compared. In the present experiment a remaining contraction (test force) of about 55% of that caused by the control force was registered. After allowing one hour for return to baseline conditions (1) 10 nM LTD4 is again injected (J) to determine the reversibility of the VR1 receptor inhibition. During steps C-F and l-J 10 μl ethanol per 100 ml PSS is present to compensate for potential vehicle effects. The experiment is concluded by adding calcium-free solution with addition of 2 mM EGTA and 20 mM caffeine for 20 min to establish the passive tension level (K). A bronchus tissue preparation is considered stable and thus fit for the evaluation of test substances if the difference in contraction between contractions D and F is less than 15 per cent.

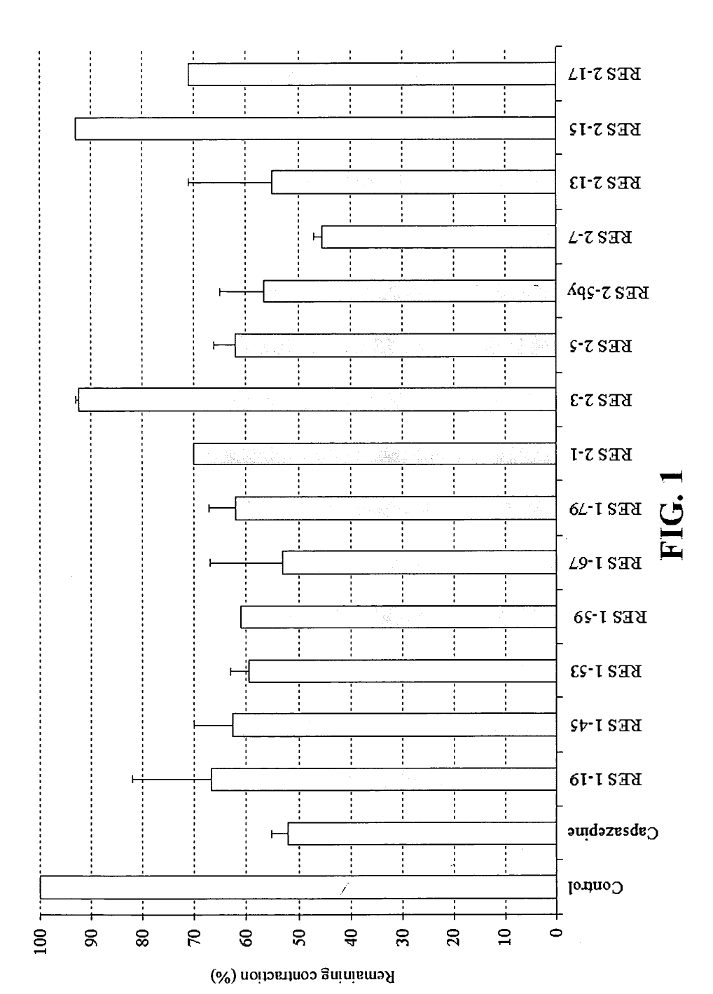

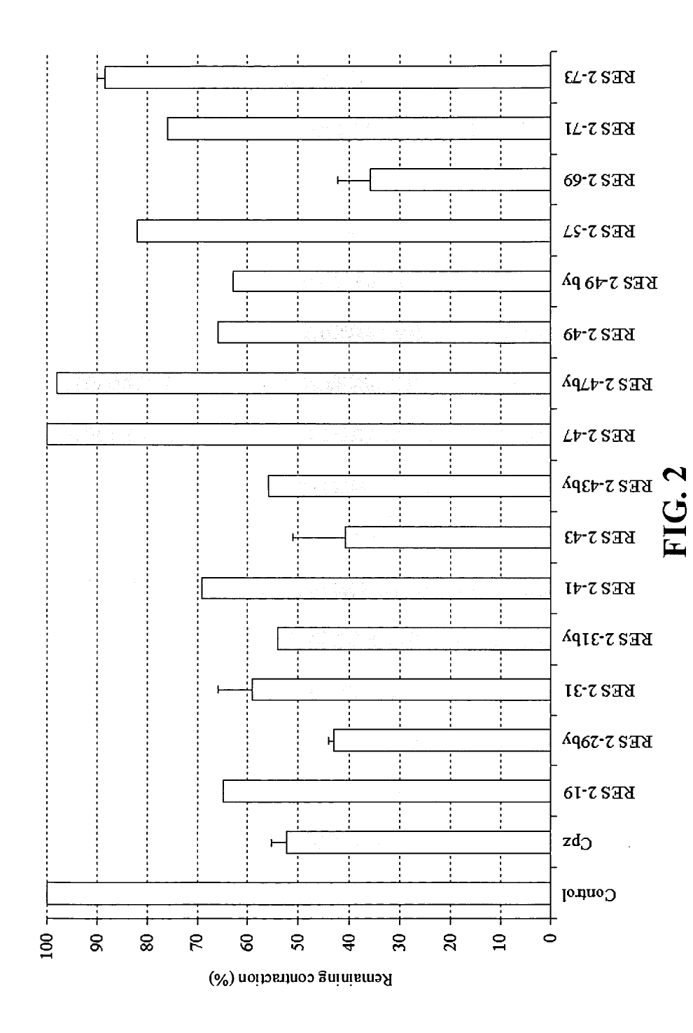

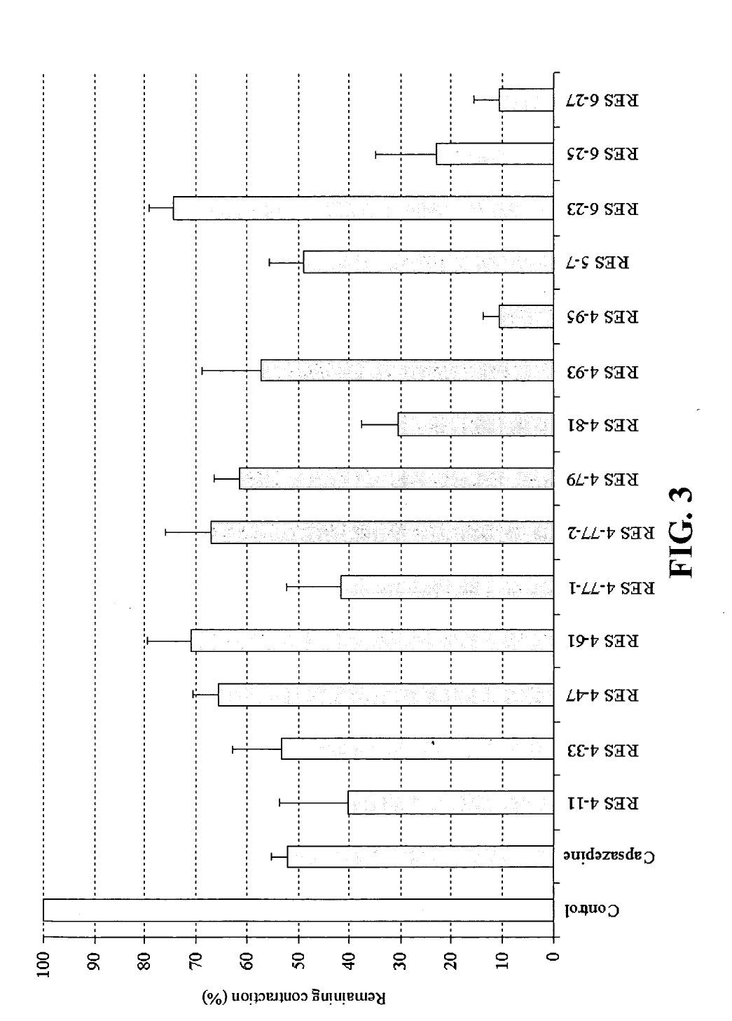

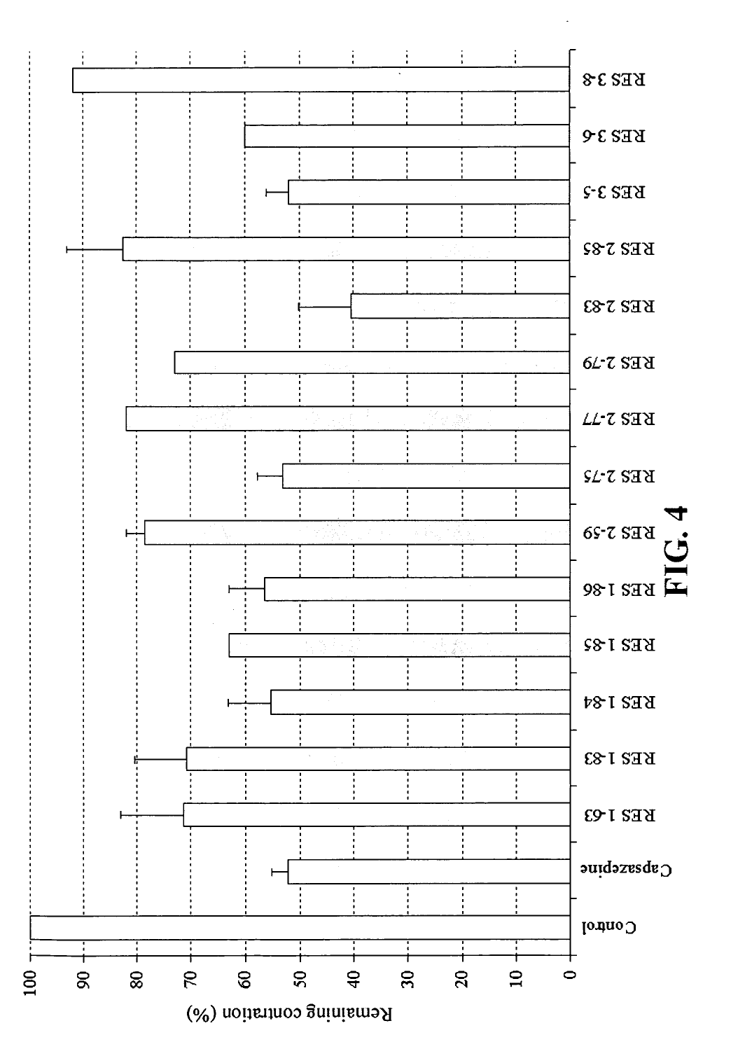

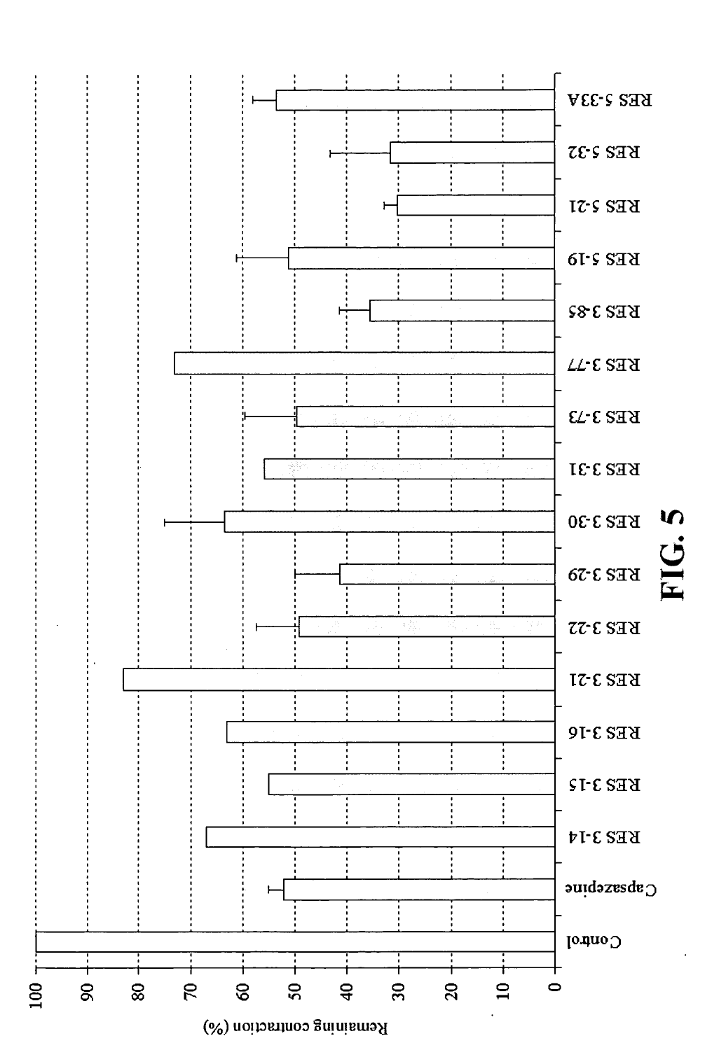

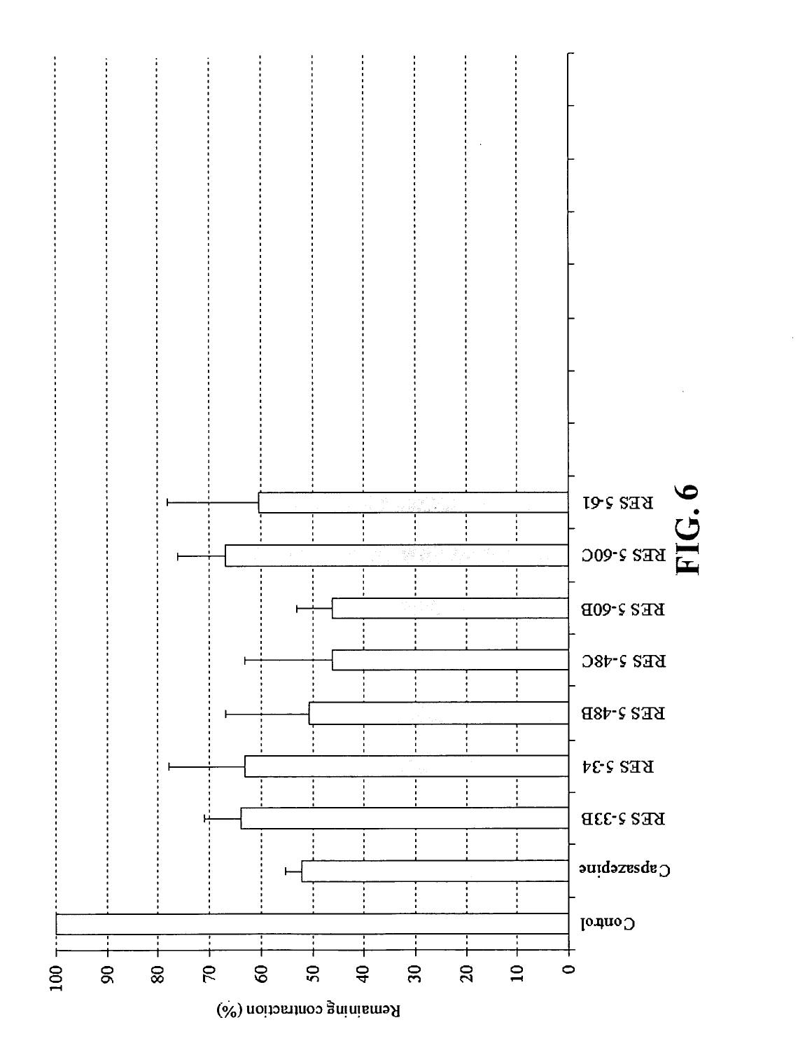

The bronchorelaxing compounds according to the invention and some prior art compounds were tested for bronchorelaxation by substituting capsazepine in the test system. The results are given in FIGS. 1-6. A measure of the bronchorelaxing capacity of a candidate substance is obtained by comparing the result (% blocking of contraction by LTD4) with that obtained with capsazepine. If the remaining contraction after exposure to a test substance is larger than after exposure to capsazepine, the test substance is less effective than capsazepine in regard of bronchorelaxing properties. If, on the other hand, the remaining contraction after exposure to a test substance is smaller than after exposure to capsazepine, the test substance is more effective than capsazepine in regard of bronchorelaxing properties.

1. A compound of the general formula (I) or a pharmaceutically acceptable acid addition salt thereof: 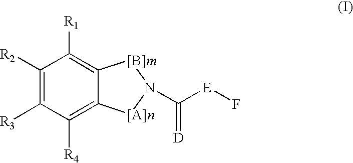
wherein
R₁-R₄ are, independent of each other H; C₁-C₆ alkyl; halogen; NR₅R₆, wherein R₅ and R₆ are, independent of each other, H, C₁-C₆ alkyl, C₂-C₆ acyl; OR₇, wherein R₇ is H, C₁-C₆ alkyl or C₂-C₆ acyl; CN; COR₈, wherein R₈ is H, C₁-C₆ alkyl or C₁-C₆ alkoxy;
A is CHR₉, wherein R₉ is H, C₁-C₆ alkyl;
n is 1-3;
B is CHR₁₀, wherein R₁₀is H, C₁-C₆ alkyl;
m is 1 or 2;
D is O or S or optionally NR₁₆, wherein R₁₆ is H, C₁-C₆ alkyl or C₂-C₆ acyl;
E is CR₁₁R₁₂ or NR₁₃, wherein R₁₁ and R₁₂ are, independent of each other, H or C₁-C₆ alkyl and wherein R₁₃ is H or C₁-C₆ alkyl;
F is C₁-C₁₈ alkyl, which is optionally mono- or di-unsaturated and is optionally substituted by alkyl, aryl, substituted aryl, heteroaryl, substituted heteroaryl, wherein, independent of each other, said C₁-C₁₈ alkyl and optional substitutents are optionally further substituted by one to three substituents independently selected from F, Cl, and Br;
with the proviso that,
if R₁ and R₂ are H, n is 2, m is 1, D is S, E is NH, F is 2-(4-chlorophenyl)ethyl or octyl, R₃ and R₄ are not both OH or OH and OCH₃;
if R₁ and R₄ are H, n is 2 or 3, m is 1, D is S, E is NH, F is 2-(4-chlorophenyl)ethyl or octyl, R₂ and R₃ are not both OH or OH and OCH₃.

2. The compound of claim 1, wherein R₉ and R₁₀ are H.

3. The compound of claim 1, wherein at least one of R₁₁, R₁₂ and R₁₃ is H.

4. The compound of claim 1, wherein R₁₁ and R₁₃ are H

5. The compound of claim 4, wherein R₉ and R₁₀ are H.

6. The compound of claim 5, wherein R₁₂ is H.

7. The compound of claim 1, wherein F is ω-(C₁-C₃)R₁₄, wherein R₁₄ is substituted or unsubstituted aryl or heteroaryl.

8. The compound of claim 7, wherein R₁₄ is mono-, di- or trisubstituted aryl or mono-, di- or trisubstituted heteroaryl, wherein said mono-, di- or trisubstitution is C₁-C₆ alkyl; aryl; heteroaryl; halogen; hydroxy, C₁-C₃ alkoxy; methylenedioxy; nitro; cyano; carboxy C₁-C₆ alkyl; R₁₅CO, wherein R₁₅ is H, C₁-C₆ alkyl, aryl; amino; alkylamino, dialkylamino; fully or partially fluorinated C₁-C₆ alkyl; with the proviso that, in case of di- or trisubstitution, the substituents are same or different.

9. The compound of claim 8, wherein at least one substituent in said mono-, di- or trisubstitution is selected from C₁-C₆ alkyl, aryl, F, Cl, Br, methyl, trifluoromethyl, nitro, and methoxy.

10. The compound of claim 8, wherein at least two substituents in said mono, di- or trisubstitution are selected from C₁-C₆ alkyl, aryl, F, Cl, Br, methyl, trifluoromethyl, nitro, and methoxy.

11. The compound of claim 1, wherein at least one of R₁-R₄ is halogen.

12. The compound of claim 11, wherein said halogen is chloro or bromo.

13. The compound of claim 11, wherein at least one of R₁-R₄ is hydroxy or methoxy.

14. The compound of claim 1, wherein at least one of R₁ and R₄ is halogen.

15. The compound of claim 14, wherein said halogen is chloro.

16. The compound of claim 1, wherein at least two of R₁-R₄ are halogen.

17. The compound of claim 16, wherein each of said halogens is independently chloro or bromo.

18. The compound of claim 16, wherein said halogen is chloro.

19. The compound of claim 16, wherein at least one of R₁ and R₄ is halogen.

20. The compound of claim 16, wherein at least one of R₁-R₄ is hydroxy or methoxy.

21. The compound of claim 20, wherein two of R₁-R₄ are, independent of each other, hydroxy or methoxy or methylenedioxy.

22. The compound of claim 1, wherein at least one of R₁ to R₄ are, independent of each other, hydroxy or methoxy or methylenedioxy.

23. The compound of claim 1, wherein at least two of R₁-R₄ are hydroxy.

24. The compound of claim 22, wherein said two hydroxy groups are in an ortho relationship to thereby form a pyrocatechol structure.

25. The compound of claim 24, wherein two of R₁-R₄ are methyl to thereby form said pyrocatechol structure which is dimethylated.

26. The compound of claim 24, wherein one of R₁ to R₄ is hydroxy and another is methoxy.

27. The compound of claim 26, wherein said hydroxy and methoxy are in an ortho relationship.

28. The compound of claim 1, wherein at least one of R₁ to R₄ is hydroxy or methoxy and at least another of R₁ to R₄ is chloro or bromo,.

29. The compound of claim 28, wherein said at least another of R₁ to R₄ is chloro.

30. The compound of claim 28, wherein said hydroxy or methoxy and said chloro or bromo are in an ortho relationship.

31. The compound of claim 1, wherein at least two of R₁ to R₄ are methoxy or comprised by methylenedioxy.

32. The compound of claim 1, wherein D is O.

33. The compound of claim 1, wherein D is S.

34. The compound of claim 1, in form of a pharmaceutically acceptable acid addition salt.

35. The compound of claim 1 selected from the group consisting of:

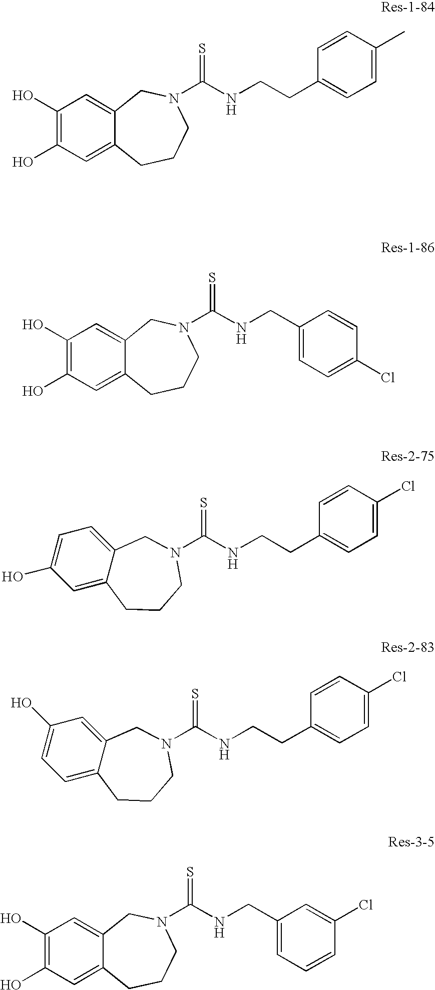

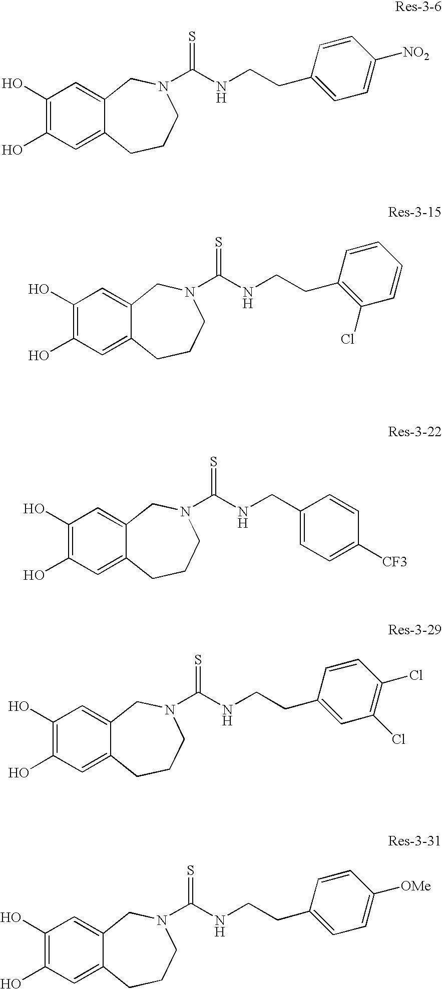

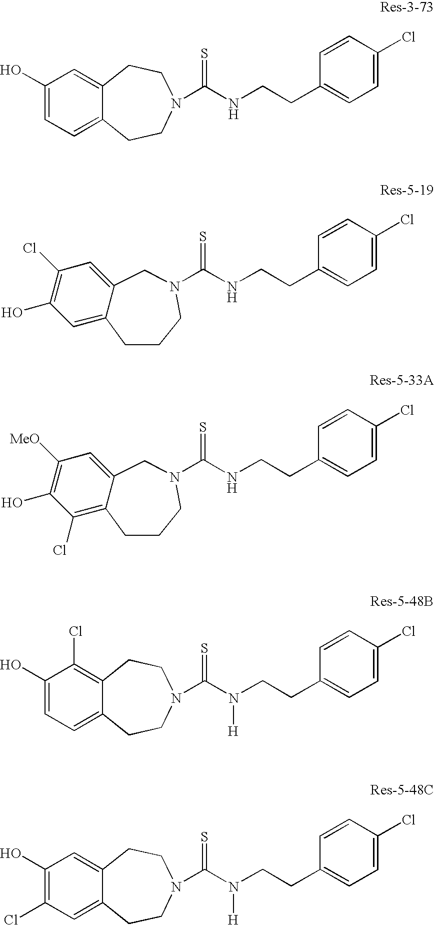

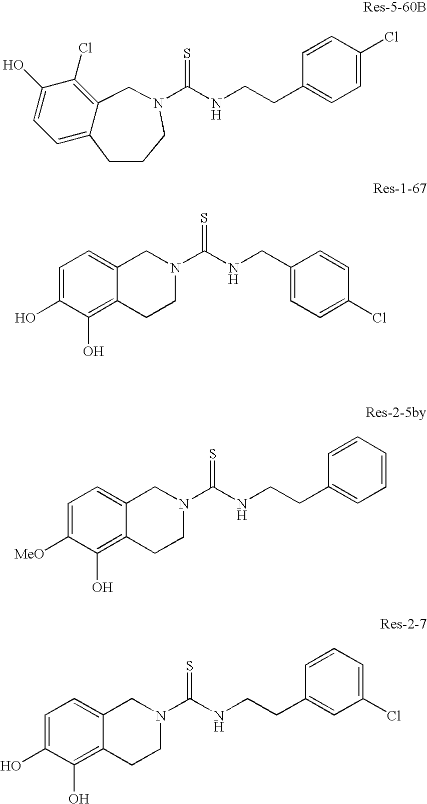

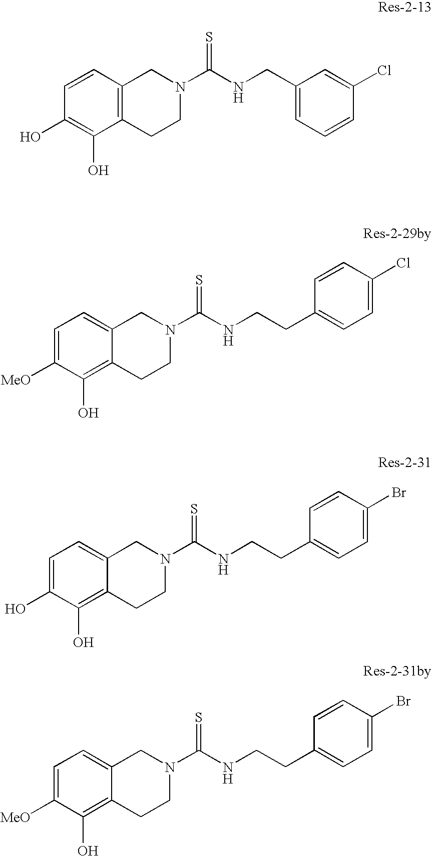

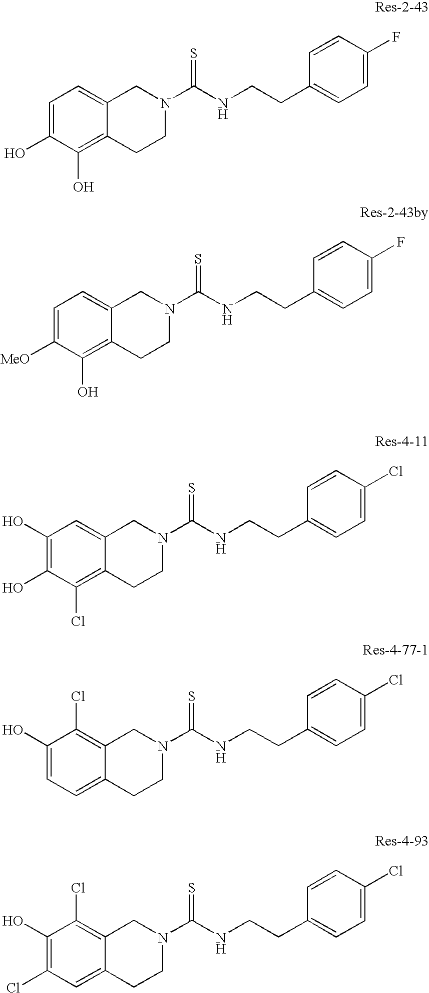

36. The compound of claim 1 selected from the group consisting of:

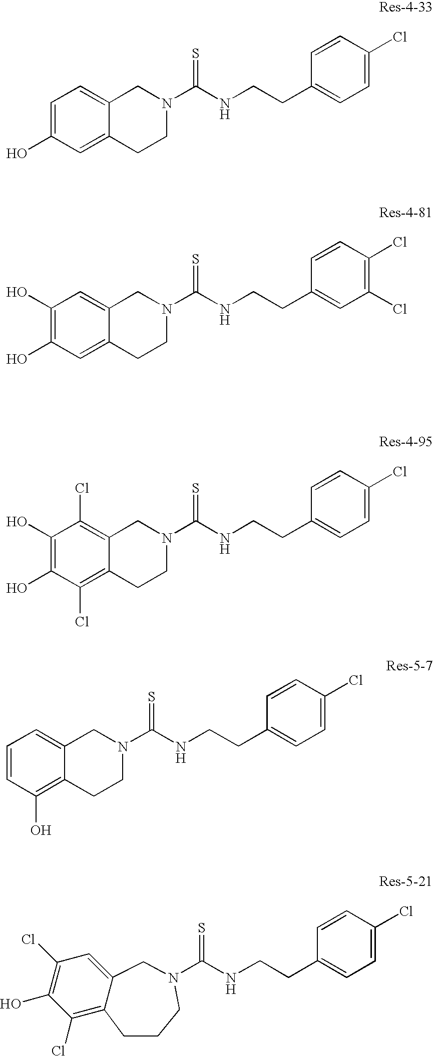

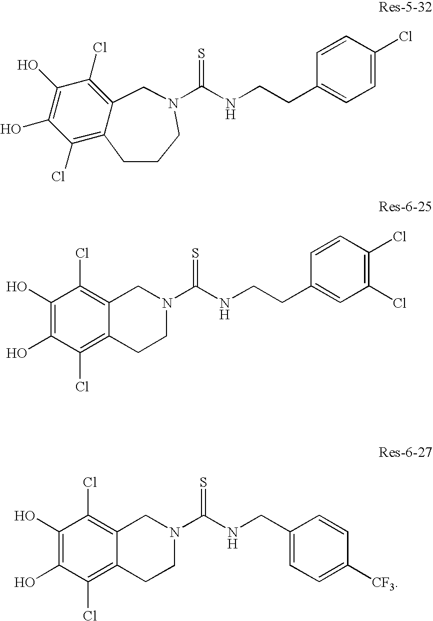

37. A compound according to claim 1 which is

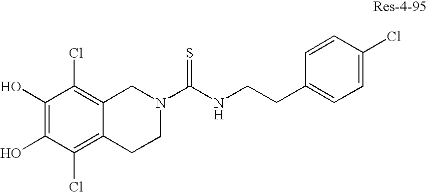

38. A pharmaceutical composition comprising an effective bronchoconstriction relaxing dose of a compound of claim 37 and a pharmaceutically acceptable carrier.

39. A pharmaceutical composition comprising an effective bronchoconstriction relaxing dose of a compound of claim 36 and a pharmaceutically acceptable carrier.

40. A pharmaceutical composition comprising an effective bronchoconstriction relaxing dose of a compound of claim 35 and a pharmaceutically acceptable carrier.

41. A pharmaceutical composition comprising an effective bronchoconstriction relaxing dose of a compound of claim 1 and a pharmaceutically acceptable carrier.

42. A method of treating or preventing pulmonary disease characterized by bronchoconstriction, comprising the administration to a person of a bronchoconstriction relaxing dose of the compound of claim 1.

43. The method of claim 39, wherein the disease is asthma, chronic obstructive pulmonary disease, bronchiectasis, cystic fibrosis, bronchiolitis or bronchopulmonary dysplasia.

44. A method of treating or preventing pulmonary disease characterized by bronchoconstriction, comprising the administration to a person of a bronchoconstriction relaxing dose of the compound of claim 35.

45. A method of treating or preventing pulmonary disease characterized by bronchoconstriction, comprising the administration to a person of a bronchoconstriction relaxing dose of the compound of claim 36.

46. A method of treating or preventing pulmonary disease characterized by bronchoconstriction, comprising the administration to a person of a bronchoconstriction relaxing dose of the compound of claim 37.
